(19) United States
(12) Patent Application Publication
(10) Pub. No.: US 2021/0287794 A1
(43) Pub. Date: Sep. 16, 2021

(54) SYSTEMS AND METHODS FOR REMOTE PRESCRIPTION OF MEDICATION-DOSING REGIMENS

(71) Applicant: Eli Lilly and Company, Indianapolis, IN (US)

(72) Inventors: Rhett Guy ALDEN, Boston, MA (US); Jeffrey Sterling ANDREWS, Arlington, MA (US); Jennal Lynn JOHNSON, Indianapolis, IN (US); James Harold PARSHALL, Westfield, IN (US); Harry Daniel TUNNELL, IV, Fort Wayne, IN (US); Scott Allen WEIGAND, Carmel, IN (US); Kristin Marie WESTERFIELD, Fishers, IN (US)

(21) Appl. No.: 17/258,208

(22) PCT Filed: Jul. 19, 2019
(86) PCT No.: PCT/US2019/042507

§ 371 Date: Jan. 6, 2021

Related U.S. Application Data
(60) Provisional application No. 62/801,217, filed on Feb. 5, 2019.
(60) Provisional application No. 62/703,563, filed on Jul. 26, 2018.

(51) Int. Cl. G16H 40/67; G16H 20/17

(52) U.S. Cl. CPC G16H 40/67; G16H 20/60; G16H 20/17

(57) ABSTRACT

Disclosed herein are systems and methods for remote prescription of medication-dosing regimens. One embodiment takes the form of a method that includes presenting, via a healthcare-professional-(HCP)-portal user interface of an HCP system, multiple medication-dosing regimens pertaining to a patient. Each presented medication-dosing regimen is associated with a respective different medication-dosing algorithm. The method also includes receiving, via the HCP-portal user interface, an HCP selection of at least one of the presented medication-dosing regimens. The method also includes transmitting, to a medical mobile application (MMA) on a mobile device associated with the patient, HCP-selected-regimen data indicative of the at least one HCP-selected medication-dosing regimen.

### FIELD OF INVENTION

The present disclosure relates to medical devices and medical-information systems, and in particular to systems and methods for remote prescription of medication-dosing regimens.

### BACKGROUND

There are myriad diseases and other medical conditions for which patients receive medical treatment. Quite often this treatment involves one or more dosing regimens of one or more medications.

### OVERVIEW

Disclosed herein are systems and methods for remote prescription of medication-dosing regimens. The present systems and methods are applicable to numerous types of medications for numerous types of diseases and other medical conditions. Patients are treated for these medical conditions by what are referred to in this disclosure as healthcare professionals (HCPs). As used herein, HCPs includes both people and/or entities that directly provide medical care to patients (e.g., doctors, nurses, nurse practitioners, physician assistants (PAs), and the like) as well as people and/or entities that do not directly provide medical care to patients but which indirectly support the provision of health-care to patients (e.g., administrative personnel, database-management personnel, other information-technology personnel, insurance and/or other payment-related personnel, and the like). In situations described herein in which an HCP prescribes a medication-dosing regimen and/or other treatment for which a prescription is required, that HCP is assumed to have the proper education, training, certification, licensing, and/or the like that would be required to issue such prescriptions. Examples of medications governed by said medication-dosing regimens include one or more therapeutic agents including but not limited to insulins, insulin analogs such as insulin lispro or insulin glargine, insulin derivatives, glucagon, glucagon analogs, glucagon derivatives, and any therapeutic agent that is capable of delivery by a medication delivery device, or that may be taken by a patient without the aid of a medication delivery device (e.g., orally or through application to the patient's skin). The medications may be formulated with one or more excipients.

One example area of applicability of the present systems and methods is diabetes (also referred to at times as diabetes mellitus), which is a general term for a group of metabolic disorders that are generally characterized by elevated levels of glucose (e.g., blood glucose, or blood sugar) in the human body for sustained periods of time. As is known in the art and in the medical community, another term for elevated glucose levels is hyperglycemia, which is typically defined as a glucose level of greater than 180 milligrams (mg) per deciliter (dL) (mg/dL), a threshold that is also expressible as the quantity 10 millimoles (mmol) per liter (L) (mmol/L). The converse condition (i.e., an insufficient level of glucose) is known as hypoglycemia, which is typically defined as a glucose level equal to or less than 70 mg/dL (3.9 mmol/L).

There are two main types of diabetes, known as type-1 diabetes and type-2 diabetes, each of which is related in a different way to insulin, which is a hormone that is central to the body's metabolic processing of glucose into energy that is either immediately used or stored for later use. Type-1 diabetes is generally considered an autoimmune disease in which the patient's immune system mistakenly attacks insulin-producing “beta cells” in the pancreas. Type-2 diabetes is generally characterized by the pancreas failing to produce enough insulin for the body's needs, or the patient's body losing its ability to respond to insulin.

Among the treatments for type-1 and/or type-2 diabetes is the subcutaneous injection of synthetic insulin. As a general matter, synthetic insulins are classified according to a number of dimensions including onset (i.e., how quickly they typically take effect), peak (i.e., how much time typically elapses until maximum efficacy is achieved), duration (i.e., the amount of time that the effect typically lasts), and concentration. The typical manner of expression for concentration is in units per milliliter (mL); as an example, a concentration of 100 units per mL would typically be expressed as U100.

In cases of type-1 and/or type-2 diabetes, there are two main categories of synthetic insulin that are typically prescribed by HCPs, typically for subcutaneous injection: basal insulin and bolus insulin. Basal insulin is also known by a number of other terms such as background insulin and long-acting insulin, and is generally taken by diabetes patients once or twice daily at regular intervals and in consistent dosages for the purpose of maintaining a certain baseline level of insulin in the patient during times such as fasting, sleeping, being in between meals, and the like. Bolus insulin also has a number of names by which it is known, including prandial insulin, mealtime insulin, and rapid-acting insulin, and is generally taken by diabetes patients on more of an as-needed basis, most typically at mealtimes, for the purpose of handling the glucose-level spikes that are typical of meal times but that can occur at other times as well.

Though certainly applicable to numerous types of medications for numerous types of diseases, conditions, and the like, the present methods and systems are intended at least in part to assist HCPs and patients with type-1 or type-2 diabetes in the management of (sometimes intensive) basal-insulin and/or bolus-insulin regimens. Indeed, although some of the examples in this disclosure generally relate to prescription and implementation of bolus-insulin dosing regimens for cases of diabetes, it should be understood that this is by way of illustration and not limitation, and is in no way to the exclusion of the present systems and methods pertaining to dosing regimens for other types of insulin (e.g., basal-insulin) and/or for other types of diseases. In general and as stated above, it should be understood that the present systems and methods are applicable to a wide variety of dosing regimens for a wide variety of diseases, conditions, and the like.

It is recognized by the inventors of the present systems and methods that managing the complexity of insulin therapy can be challenging, especially on the patient side, but also on the HCP side. The present systems and methods address a number of technical problems with the prior art, including the technical problem that prior medical-device and medical-technology implementations in general do not provide a mechanism by which an HCP can select and even customize at least one medication-dosing regimen (e.g., a bolus-insulin-dosing regimen) out of a plurality of pre-determined and pre-programmed dosing regimens for a particular patient and then further prescribe that at least one regimen to that patient over a data network by interacting remotely with an application running on a mobile device associated with the patient. In accordance with some embodiments of the present systems and methods, logic for the plurality of pre-determined and pre-programmed dosing regimens can be provided with the application running on the patient's mobile device, but may be initially locked so as to be inaccessible to the patient. Further in accordance with some embodiments, the HCP can remotely unlock at least one particular HCP-selected regimen out of the plurality of pre-programmed dosing regimens for that patient on that mobile device, which then can provide instructions to the patient for implementing the at least one regimen, and in some cases direct one or more additional medical devices (that is or are also associated with the patient) to carry out some or all of the at least one HCP-selected dosing regimen. The present systems and methods represent a technical solution to at least that technical problem. And it is respectfully noted that this paragraph in no way implies that other technical solutions to other technical problems are not represented by this disclosure.

One embodiment takes the form of a method that includes presenting, via an HCP-portal application executing on an HCP system, multiple medication-dosing regimens pertaining to a patient. Each presented medication-dosing regimen is associated with a respective different medication-dosing algorithm. The method also includes receiving, via the HCP-portal user application, an HCP selection of at least one of the presented medication-dosing regimens. The method also includes transmitting, to a medical mobile application (MMA) on a mobile device associated with the patient, HCP-selected-regimen data indicative of the at least one HCP-selected medication-dosing regimen.

Another embodiment takes the form of a connected-care server system that includes one or more servers that each include one or more communication interfaces, one or more processors, and data storage. In at least some embodiments, the connected-care server system is a connected-care-cloud (C3) server system, and each of the one or more servers are cloud-servers. The data storage of the one or more servers collectively contains instructions executable by one or more of the processors of the one or more servers for causing the connected-care server system to carry out a set of server-system functions that includes the functions that are listed in the preceding paragraph. Yet another embodiment takes the form of one or more non-transitory computer-readable media (CRM) having stored thereon instructions that, when executed by one or more processors, are operable to cause the one or more processors to carry out a set of functions that includes the functions that are listed in the preceding paragraph. In connection with any embodiment, a CRM could take the form of any non-transitory data-storage medium mentioned herein and/or any other non-transitory data-storage medium deemed suitable by those of skill in the art for a given implementation. Examples include any suitable forms of memory (including both volatile and non-volatile memory), disk storage, optical storage, and/or the like.

Another embodiment takes the form of a method that includes receiving, at an MMA that is executing on a mobile device that is associated with a patient, HCP-selected-regimen data that is indicative of an HCP selection of an HCP-selected medication-dosing regimen from among a plurality of medication-dosing regimens, each of which is associated with a respective different medication-dosing algorithm. The HCP selection had been made for the patient by the HCP via an HCP-portal application that is executing on a HCP system. The method also includes unlocking the indicated HCP-selected medication-dosing regimen on the MMA responsive at least in part to having received the HCP-selected-regimen data.

Another embodiment takes the form of a mobile device that is associated with a patient. The mobile device includes a mobile-device communication interface, a mobile-device processor, and mobile-device data storage. The mobile-device data storage contains instructions executable by the mobile-device processor for causing the mobile device to execute an MMA for carrying out a set of MMA functions that includes the functions that are listed in the preceding paragraph. Yet another embodiment takes the form of one or more non-transitory CRMs having stored thereon instructions that, when executed by one or more processors, are operable to cause the one or more processors to carry out a set of functions that includes the functions that are listed in the preceding paragraphs.

Another embodiment takes the form of a system that includes the above-described connected-care server system and the above-described mobile device. At least one embodiment further includes the above-described HCP system.

Another embodiment takes the form of a method comprising: presenting, by a processor on a mobile device associated with a patient, a plurality of panels simultaneously on a visual display of the mobile device, wherein the plurality of panels comprise: a first panel that displays a number of units of insulin to be administered to the patient, and a second panel that displays an amount of carbohydrates expected to be covered by the number of units of insulin displayed by the first panel; receiving at the mobile device user input representative of a requested adjustment to the number of units of insulin displayed by the first panel; and updating, by the processor, each panel of the plurality of panels according to the requested adjustment to the number of units of insulin displayed by the first panel, wherein the updating includes displaying an adjusted number of units of insulin by the first panel and an adjusted amount of carbohydrates expected to be covered by the adjusted number of units of insulin with the second panel.

In some embodiments, the plurality of panels may further comprise a panel that displays an indication of a meal size corresponding to the amount of carbohydrates displayed by the second panel, and wherein the updating includes displaying an indication of an adjusted meal size corresponding to the adjusted amount of carbohydrates. Alternatively, or in addition, the plurality of panels may further comprise a panel that displays a spectrum that displays a range of meal sizes between a small meal size and a large meal size, and an indicator that indicates where the amount of carbohydrates displayed by the second panel falls on the spectrum. Alternatively, or in addition, the plurality of panels may further comprise a panel that indicates whether the amount of carbohydrates displayed by the second panel corresponds to a small meal size, a medium meal size, or a large meal size. Alternatively, or in addition, the plurality of panels may further comprise a panel that displays a drop in the patient's glucose level that is expected to result from administration of the number of units of insulin displayed by the first panel, and wherein the updating includes displaying an adjusted drop in the patient's glucose level that is expected to result from administration of the adjusted number of units of insulin.

In some embodiments, the method may further comprise receiving at the mobile device a maximum carbohydrate threshold for a medium meal size and a minimum carbohydrate threshold for a medium meal size, wherein one of the plurality of panels indicates whether the number of carbohydrates displayed by the second panel corresponds to a small meal size, a medium meal size, or a large meal size based on the received maximum carbohydrate threshold and minimum carbohydrate threshold. The method may also further comprise receiving at the mobile device an insulin-to-carbohydrate ratio (ICR) associated with the patient, wherein the amount of carbohydrates displayed by the second panel is calculated by the processor based on the number of units displayed by the first panel and the received ICR. The method may also further comprise receiving at the mobile device an insulin sensitivity factor (ISF) associated with the patient, wherein the drop in the patient's glucose level displayed by one of the plurality of panels is calculated based on the number of units of insulin displayed by the first panel and the received ISF.

A number of variations and permutations of the above-listed embodiments are described herein. Moreover, it is expressly noted that any variation or permutation that is described in this disclosure can be implemented with respect to any type of embodiment. For example, a variation or permutation that is primarily described in connection with a method embodiment could just as well be implemented in connection with a system embodiment and/or a CRM embodiment. Furthermore, this flexibility and cross-applicability of embodiments is present in spite of any slightly different language (e.g., process, method, steps, functions, set of functions, and the like) that is used to describe and/or characterize such embodiments.

### BRIEF DESCRIPTION OF THE DRAWINGS

A more detailed understanding may be had from the following description, which is presented by way of example in conjunction with the following drawings, in which like reference numerals are used across the drawings in connection with like elements.

FIG. 1 depicts an example communication context that includes an example HCP system, an example health information technology (IT) system, an example mobile device associated with an example patient and in communication with multiple example personal medical devices that are also associated with the example patient, an example server system that includes one or more example servers (e.g., connected-care cloud servers) that are communicatively interconnected with one another via an example network, and an example administrator-portal system, in accordance with at least one embodiment.

FIG. 2 depicts an example physical architecture of the example HCP system of FIG. 1, in accordance with at least one embodiment.

FIG. 3 depicts an example physical architecture of the example mobile device of FIG. 1, in accordance with at least one embodiment.

FIG. 4A depicts an example physical architecture of an example one of the servers in the example server system of FIG. 1, in accordance with at least one embodiment.

FIG. 4B depicts an example physical architecture of the example administrator-portal system of FIG. 1, in accordance with at least one embodiment.

FIG. 5 depicts an example functional architecture of the example server system of FIG. 1, in accordance with at least one embodiment.

FIG. 6 depicts various entities in an example communication-and-processing ecosystem, in accordance with at least one embodiment.

FIG. 7 depicts a first example method, which may be carried out by the server system of FIG. 1, in accordance with at least one embodiment.

FIG. 8 depicts a second example method, which may be carried out by the mobile device of FIG. 1 executing an example MMA, in accordance with at least one embodiment.

FIGS. 9 through 18 depict various example screenshots for display via the example HCP system of FIG. 1, in accordance with at least one embodiment.

FIG. 19 depicts a flowchart illustrating an exemplary process for calculating, presenting, and editing a recommended dose of insulin implemented by a MMA, in accordance with at least one embodiment.

FIG. 20 depicts an exemplary screenshot from a MMA in which the MMA automatically synchronizes with a patient's glucose sensor, in accordance with at least one embodiment.

FIGS. 21A, 21B, and 21C depict exemplary screenshots from a MMA in which the MMA receives a patient's glucose level, in accordance with at least one embodiment.

FIGS. 22A, 22B, 22C, and 22D depict exemplary screenshots from a MMA used by the MMA for a daily titration dosing regimen, in accordance with at least one embodiment.

FIGS. 23A, 23B, 23C, and 23D depict exemplary screenshots from a MMA used by the MMA for a fixed-dose dosing regimen, in accordance with at least one embodiment.

FIGS. 24A, 24B, 24C, and 24D depict exemplary screenshots from a MMA used by the MMA to receive a number of carbohydrates for a carb bolus calculator dosing regimen, in accordance with at least one embodiment.

FIGS. 25A, 25B, 25C, and 25D depict exemplary screenshots from a MMA used by the MMA to receive a number of units of mealtime insulin for a carb bolus calculator dosing regimen, in accordance with at least one embodiment.

FIGS. 26A, 26B, 26C, and 26D depict exemplary screenshots from a MMA used by the MMA for a carb meal-size-based calculator dosing regimen, in accordance with at least one embodiment.

FIGS. 27A, 27B, and 27C depict exemplary screenshots from a MMA used by the MMA to present and/or edit a recommended dose of insulin, in accordance with at least one embodiment.

FIG. 28 depicts an exemplary user-interface display having four panels of information related to a presented insulin dose, in accordance with at least one embodiment.

FIG. 29 depicts an exemplary sequence of user-interface displays that show how the four panels of information update as the presented insulin dose is adjusted, in accordance with at least one embodiment.

FIG. 30 depicts an example embodiment in which the user-interface of FIGS. 28 and 29 is displayed on a connected drug-delivery device, in accordance with at least one embodiment.

FIG. 31 depicts a flow-chart illustrating an exemplary process executed by a mobile device to display and update the user-interface of FIGS. 28, 29, and 30, in accordance with at least one embodiment.

For the purposes of promoting an understanding of the principles of the present disclosure, reference is made below to the embodiments illustrated in the drawings, which are described below. The embodiments disclosed herein are not intended to be exhaustive or to limit the present disclosure to the precise form disclosed in the following detailed description. Rather, the embodiments are chosen and described so that others skilled in the art may utilize their teachings. Therefore, no limitation of the scope of the present disclosure is thereby intended.

In some instances throughout this disclosure and in the claims, numeric modifiers such as first, second, third, and fourth are used in reference to various components, data such as various identifiers, and/or other elements. Such use is not intended to denote or dictate a specific or required order of the elements. Rather, this numeric terminology is used to assist the reader in identifying the element that is being referenced and in distinguishing that element from other elements, and should not be narrowly interpreted as insisting upon any particular order.

Moreover, before proceeding with this detailed description, it is noted that the entities, arrangements, and the like that are depicted in—and described in connection with—the various drawings are presented by way of example and not by way of limitation. As such, any and all statements or other indications as to what a particular drawing “depicts,” what a particular element or entity in a particular drawing “is” or “has,” and any and all similar statements—that could in isolation and out of context be read as absolute and therefore limiting—can only properly be read as being constructively (unless actually) preceded by a clause such as “In at least one embodiment, . . . .” And it is for reasons akin to brevity and clarity of presentation that this implied leading clause is not repeated ad nauseum in this detailed description.

### DETAILED DESCRIPTION OF THE DRAWINGS

### I. Example Architecture

A. Example Communication Context

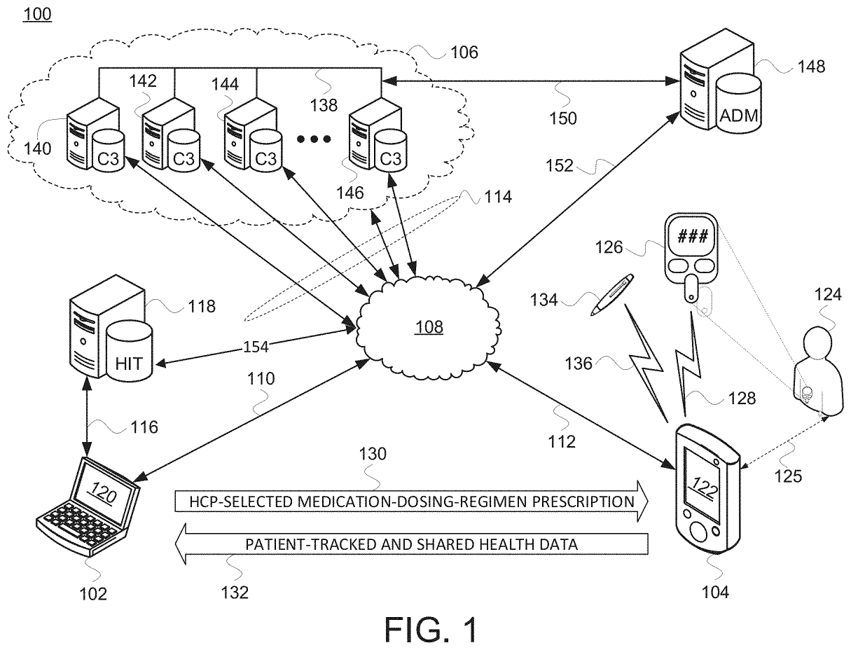

FIG. 1 depicts an example communication context 100 that includes an example HCP system 102, an example health IT system 118, an example mobile device 104 associated with an example patient 124 and in communication with multiple example personal medical devices (in this depiction, an example glucose meter 126 and an example connected injection device 134) that are also associated with the example patient 124, an example server system 106 that includes one or more example servers 140-146 that are communicatively interconnected with one another via an example private network 138, and an example administrator-portal system 148, in accordance with at least one embodiment. Also depicted in FIG. 1 are a data network 108, communication links 110, 112, 114, 116, 128, 136, 150, 152, and 154; a display 120 of the HCP system 102; and a display 122 of the mobile device 104. Further displayed are an association arrow 125, a conceptual-information-flow arrow 130, and a conceptual-information-flow arrow 132, both of which are described below. It should be understood that the entities and the arrangement and interconnection thereof that are depicted in FIG. 1 are provided for illustration and by way of example and not limitation, and that other entities, arrangements, and interconnections could be implemented as well as deemed suitable by those of skill in the art for a given context.

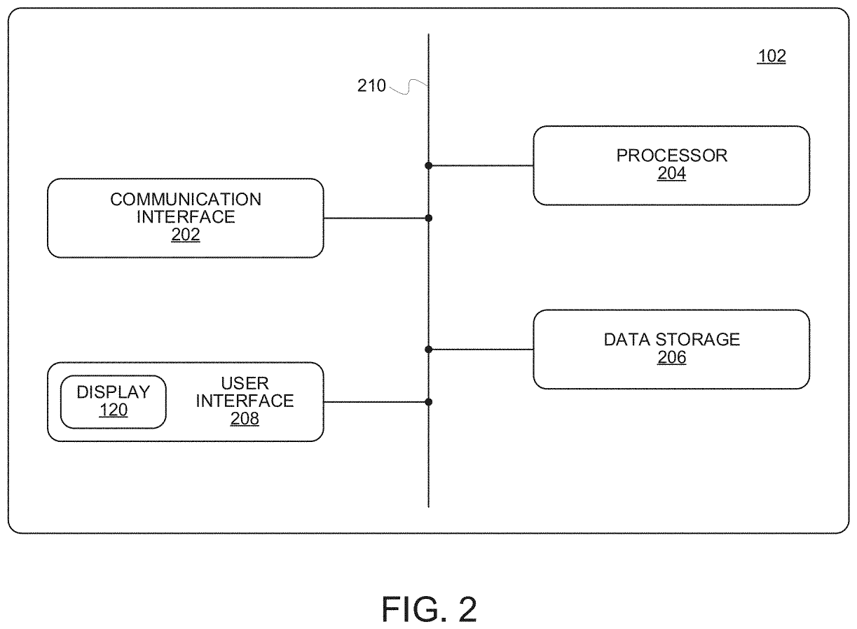

The HCP system 102 (including the display 120) is described from a physical-architecture standpoint more fully below in connection with FIG. 2, but in general could take the form of any computing device that is equipped, configured, and programmed to carry out the HCP-portal functions described herein. Some options for the HCP system 102 include a desktop computer, a laptop computer, a tablet computer, a mobile device such as a smartphone, or a device that interacts with the HCP using voice, augmented reality (AR) or virtual reality (VR). In some embodiments, HCP system 102 is any electronic device capable of supporting and running an Internet browser (i.e., a web browser—perhaps as a standalone application, perhaps as a capability of another application, to name but a few examples). As depicted in FIG. 1, the HCP system 102 is communicatively connected to the health IT system 118 via the communication link 116 and to the data network 108 via the communication link 110. Although communication link 116 is depicted as being separate from data network 108, it should be understood that in some instances, all or part of link 116 may traverse at least a portion of data network 108. In some instances, the HCP system 102 may communicate with HIT system 118 indirectly via the data network 108, instead of through a direct link 116 as depicted in FIG. 1. Among the typical users of the HCP system 102 are HCPs with prescribing roles (e.g., doctors, physician assistants (PAs), nurse practitioners, and the like), as well as non-prescribing HCPs who are part of a care team, and users associated with integrated delivery networks (IDNs), among other examples.

In various different embodiments, the HCP system 102 provides and supports functions such as prescribing medication-dosing regimens (e.g., insulin-dosing regimens such as basal-insulin-dosing regimens, bolus-insulin-dosing regimens, and/or the like), modifying existing dosing-regimen prescriptions, setting and/or modifying dosing-regimen parameters, and viewing individual patient data, among other examples that could be listed here. The prescription and parameter-setting with respect to dosing regimens by an HCP via the HCP system 102 assists patients in determining medication doses. Among other functions, the HCP system 102 provides the HCP with the capability to download patient EHR data from the health IT system 118 via the communication link 116 and view the EHR data in the HCP portal, view a selection of available medication-dosing regimens, select a dosing regimen to prescribe to the patient, and assign values to required parameters for the selected dosing regimen (such as starting dose, insulin-to-carb ratio, and the like, in the example case of an insulin-dosing regimen). The HCP system 102 further provides data-visualization features that enable the HCP users to review patient historical data. In various embodiments, this patient historical data is viewable in chart form, graphical form, tabular form, and/or one or more other forms deemed suitable by those of skill in the art for a given implementation.

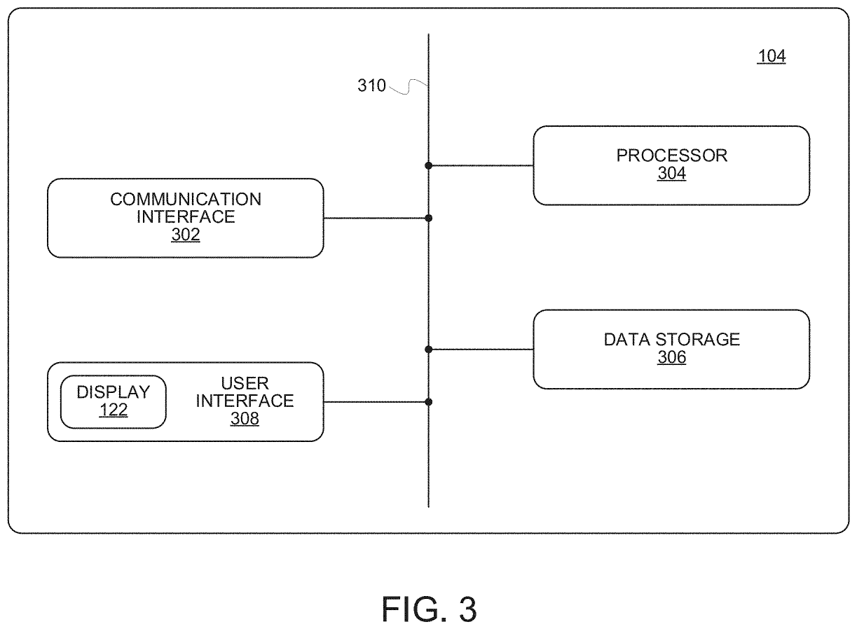

The mobile device 104 (including the display 122) is described from a physical-architecture standpoint more fully below in connection with FIG. 3, but in general could take the form of any computing device that is equipped, configured, and programmed to carry out the mobile-device functions described herein. Some options for the mobile device 104 include a cell phone, a smart phone, a personal digital assistant (PDA), a tablet computer, a laptop computer, a wearable device, a mobile device that interacts with its user via voice or virtual reality, and the like. As depicted in FIG. 1, the mobile device 104 is communicatively connected to the glucose meter 126 via the communication link 128, to the connected injection device 134 via the communication link 136, and to the data network 108 via the communication link 112. As is depicted by the double-ended-and-dashed association arrow 125, the mobile device 104 is associated—e.g., by a subscription account, ownership, possession, and/or in one or more other ways—with the patient 124.

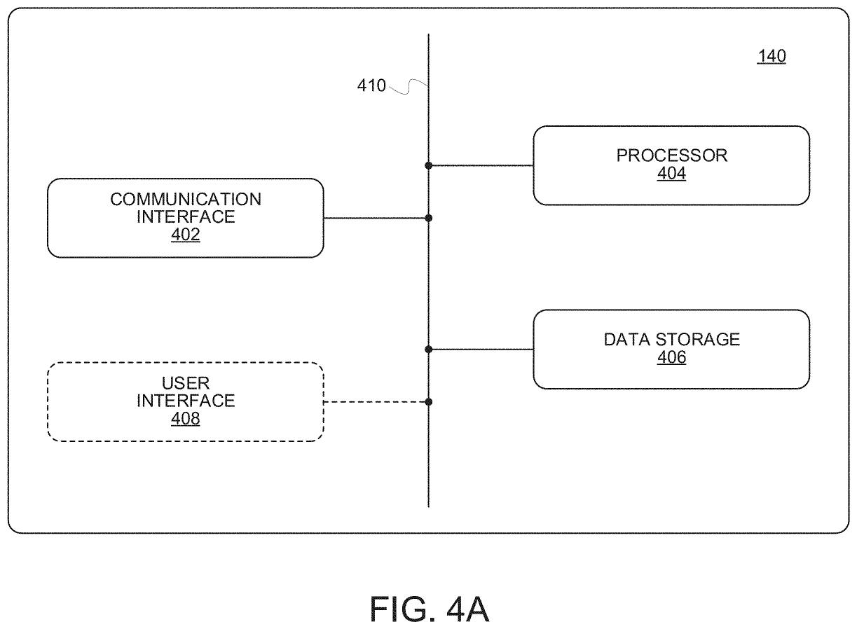

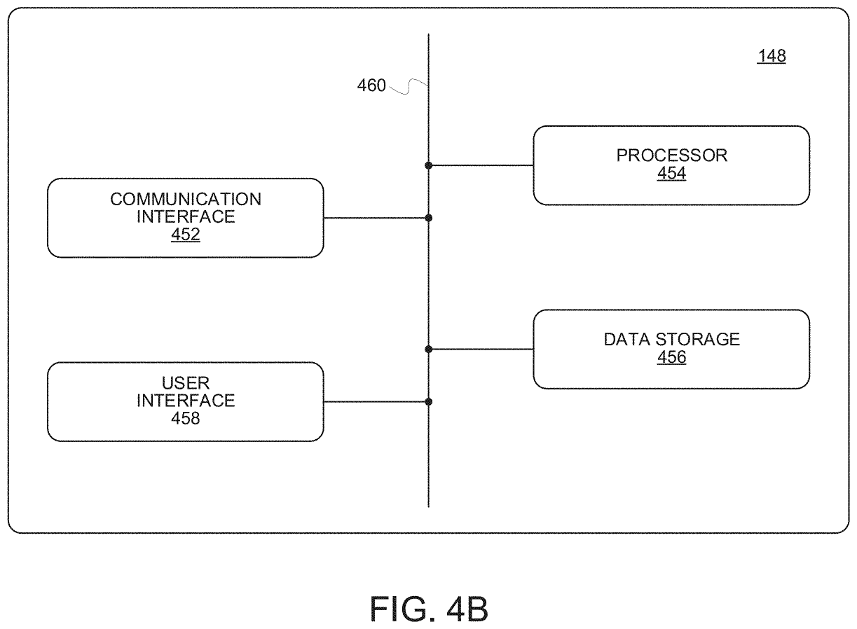

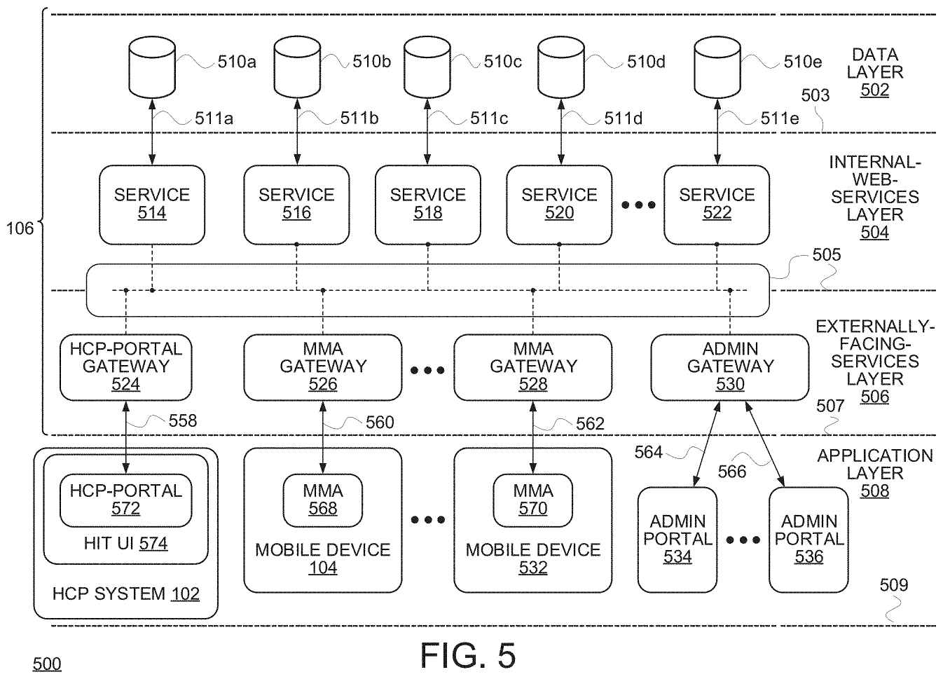

With respect to the C3-server system 106, an example one of the C3 servers 140-146 is described from a physical-architecture standpoint more fully below in connection with FIG. 4A. Moreover, the C3-server system 106 as a whole is described from a functional-architecture (e.g., software-architecture) perspective more fully below in connection with FIG. 5. In general, however, the C3-server system 106 could take the form of any set of one or more servers that are collectively equipped, configured, and programmed to collectively carry out the C3-server-system functions described herein. As depicted in FIG. 1, the C3-server system 106 is communicatively connected to the data network 108 via the communication link 114.

It is noted that, as depicted in FIG. 1, each respective C3 server 140-146 in the C3-server system 106 could have its own respective communication link 114 with the data network 108; also or instead, a single communication link 114 could communicatively connect the C3-server system 106 with the data network 108, perhaps via a firewall, a network access server (NAS), and/or the like. For clarity of presentation, the one or more communication links 114 are referred to as “the communication link 114” in the balance of this disclosure. Moreover, as is also depicted in FIG. 1, the respective C3 servers 140-146 are communicatively interconnected with one another via a private network 138, which could take the form of or include a local area network (LAN), a virtual private network (VPN), and/or one or more other options deemed suitable by those of skill in the art for a given implementation. In some embodiments, the depicted network 138 would not exist as a separate network as currently depicted—in such embodiments, the respective C3 servers 140-146 communicate with one another via their links to data network 108. The communicative connectivity between the C3-server system 106 and the administrator-portal system 148 is discussed below.

As a general matter, the C3-server system 106 functions as “the cloud” as that term is used in the art with respect to entities such as the HCP system 102, the health IT system 118, and the mobile device 104 (and in particular to one or more applications such as one or more MMAs executing on the mobile device 104). In some embodiments, a subset of C3 servers 140-146 may be dedicated to serving at least one of the HCP system 102 (e.g., to provide the HCP portal application 572, described in detail below), the health IT system 118, and the mobile device 104. In some embodiments, however, each of the C3 servers 140-146 may be capable of serving any of these three entities. Regardless of the specific architectural implementation, the C3-server system 106 functions as at least one cloud with particular purposes for those entities. As such, in at least one embodiment, all communication to and from the C3-server system 106 with any one or more other entities is secure communication—as examples, such communication could be encrypted and/or signed as is known in the art. Such communication could be inside a tunnel such as a VPN, among other communication-security and data-security options that could be implemented as deemed suitable by those of skill in the art in various contexts.

In various different embodiments, and as is further described below, the C3-server system 106 provides and supports functions—for the mentioned entities and perhaps others—such as secure and reliable transfer of information and data (related to, e.g., prescriptions) between the HCP system 102 and MMAs running on mobile devices associated with patients, data storage, management of relationships between patients and HCPs, IDNs, and the like, and in some embodiments, instead or in addition, provides and supports one or more other functions deemed suitable by those of skill in the art for a given implementation. Moreover, in some embodiments, the C3-server system 106 facilitates data sharing that involves payers (e.g., insurance companies); in some such embodiments, such data sharing with payer entities is conditional upon the associated patients opting in to allow such communication. And other examples of provided and supported functions could be listed here as well.

The administrator-portal system 148 is described from a physical-architecture standpoint more fully below in connection with FIG. 4B, but in general could take the form of any computing device that is equipped, configured, and programmed to carry out the administrator-portal-system functions described herein. Some options for the administrator-portal system 148 include a desktop computer, a laptop computer, a tablet computer, a workstation, and the like. As depicted in FIG. 1, the administrator-portal system 148 in at least one embodiment is operable to communicate with the C3-server system 106 (and in particular in the depicted example via the private network 138) via the communication link 150. In other embodiments, as is also depicted in FIG. 1, the administrator-portal system 148 is operable to communicate with the HCP system 102, the mobile device 104, and/or the C3-server system 106 via the data network 108. And in some embodiments, the administrator-portal system 148 is operable to do both.

As a general matter, in various different embodiments, the administrator-portal system 148 provides various services with respect to the HCP-portal 102, an MMA executing on the mobile device 104, and/or the C3-server system 106. One example category of such services are those that pertain to user management, login, access level, and the like. In at least one embodiment, a user with sufficient permissions can use the administrator-portal system 148 to change and/or manage various settings of the HCP-portal 102, an MMA executing on the mobile device 104, and/or the C3-server system 106. In some cases, changes made via the administrator-portal system 148 affect only a single MMA, a single user, a single HCP, and/or the like; in other cases, such changes affect multiple MMAs, multiple user accounts, multiple HCPs, and/or the like. For example, an Integrated Delivery Network (IDN) (e.g., a network of health care organizations) may be provided with an administrator-portal system 148 that governs accounts of patients enrolled in the IDN. In some embodiments, the administrator-portal system 148 is operable to roll out updates, upgrades, and the like. In some embodiments, the administrator-portal system 148 is operable to manage individual HCP accounts, individual patient accounts, and/or the like. And certainly numerous other example administrative functions for which the administrator-portal system 148 could be used could be listed here.

In the example situation that is depicted in FIG. 1, the data network 108 is communicatively connected with the HCP system 102 via the communication link 110, with the mobile device 104 via the communication link 112, with the C3-server system 106 via the communication link 114, and with the administrator-portal system 148 via the communication link 152. In at least one example scenario, the data network 108 includes one or more Internet Protocol (IP) networks such as the worldwide network of networks typically referred to broadly as the Internet, one or more public IP networks, one or more private (e.g., corporate) IP networks, and/or one or more IP networks of any other type deemed suitable by those of skill in the art for a given implementation. Entities that communicate via the data network 108 may be identified by an address such as an IP address (e.g., an IPv4 address or an IPv6 address). It is not required that the data network 108 be or include an IP network, however, as this is merely an example.

As used herein, a “communication link” includes one or more wired-communication (e.g., Ethernet, Universal Serial Bus (USB), and/or the like) links and/or one or more wireless-communication (e.g., cellular, Wi-Fi, Bluetooth, and/or the like), and may also include any suitable number of relay devices such as routers, switches, bridges, and/or the like. The communication link 112 in particular may include at least one wireless-communication link to facilitate two-way data communication with the mobile device 104. Moreover, either or both of the communication links 128 and 136 may take the form of or at least include at least one of a near-field communication (NFC) link, a Bluetooth link, a radio-frequency identification (RFID) link, a direct radio frequency (RF) link, and/or one or more other types of wireless-communication links. Moreover, the communication links 128 and 136 could but need not be point-to-point links between (i) the mobile device 104 and (ii) the glucose meter 126 and connected injection device 134, respectively: in some embodiments, one or both of the communication links 128 and 136 are an indirect link via, e.g., a Wi-Fi or ZigBee router, or a cellular network or tower (not depicted). And other implementation examples could certainly be listed here as well.

In the example scenario that is depicted in FIG. 1, the health IT system 118 is communicatively connected to the HCP system 102 via the communication link 116, and in general could take the form of one or more servers. The health IT system 118 may or may not have its own local user interface, such as its own monitor display, keyboard, mouse, touchscreen, or other user interface. In various different embodiments, the health IT system 118 optionally provides and supports secure maintenance of, secure storage of, and secure access to patient Electronic Health Records (EHRs—also referred to as Electronic Medical Records or EMRs in the industry), perhaps among other functions deemed suitable for a health IT system by those of skill in the art. Moreover, health IT system 118 is communicatively coupled to data network 108 via communication link 154. Health IT system 118 may also communicate with C3-server system 106 via data network 108. In some embodiments, the health IT system 118 interfaces with the C3-server system 106 and/or HCP system 102 via a standardized protocol, such as the Fast Healthcare Interoperability Resources (FHIR) protocol, or the Substitutable Medical Applications and Reusable Technologies (SMART) protocol, as examples.

In the example scenarios described herein, the patient 124 has been diagnosed and is being treated by an HCP—that is associated with the HCP system 102—with diabetes, though this is purely by way of example and not limitation. In the described embodiments, both the mobile device 104 (and at least one MMA running thereon), the glucose meter 126, and the connected injection device 134 are each associated with the patient 124, and in at least that way with one another. The above-mentioned association arrow 125 is intended to represent a general association and a user-interface-level interaction of the patient 124 with the mobile device 104.

It is further noted that while FIG. 1 shows mobile device 104 being connected to both glucose meter 126 and connected injection device 134, in some cases, only one of the glucose meter 126 and the connected injection device 134 may be present in various different scenarios, and that both are not required. Indeed, some scenarios involve no additional medical devices connected to mobile device 104 at all, such as is the case with systems that require patients to separately and manually measure glucose levels and/or log dosage information, or as is the case with medication that is taken by mouth and where no devices are required to monitor or otherwise test one or more levels, conditions, or other parameters of the patient 124. In some cases, a patient may connect more than one glucose meter, and/or more than one connected injection device, to the MMA—for example, a patient may have one glucose meter and/or injection device at home, and one glucose meter and/or injection device at work. The MMA may be configured to support multiple connections of each. Moreover, in some cases, one or more connected medical devices other than a glucose meter and/or a connected injection device are present. Some examples include a blood-pressure monitor, a pulse/oxygen monitor, and/or the like. And certainly many other examples could be listed here.

The glucose meter 126 is associated with the patient 124 for medical, diabetes-treatment purposes, and is communicatively connected with the mobile device 104 via the above-described communication link 128. The glucose meter 126 could include a blood-glucose meter (BGM), a continuous glucose meter (CGM), a flash glucose monitor (FGM), or other devices that measure the patient 124's blood glucose or other sources of glucose levels (e.g., contact lens devices, or devices that use near IR radiation to measure glucose levels). A BGM takes discrete spot measurements of the patient's blood-glucose level (e.g., by pricking the patient's finger to conduct spot measurements of the patient's capillary whole blood glucose level). Both CGM and FGM use sensors to measure interstitial glucose. A CGM system may include a sensor, transmitter and receiver/app. A CGM may take more frequent (i.e., more continuous) measurements of the patient's interstitial glucose levels, and may optionally be continuously worn by the patient for relatively extended periods of time (e.g., several hours or days at a time). One example of such a CGM is the G6 sensor manufactured by Dexcom, Inc. A FGM system may comprise a sensor worn on or inserted into a portion of the patient's body, and a reader (e.g., a handheld reader) that, when activated or when placed in close proximity to the sensor, receives a glucose level reading from the sensor. One example of such a FGM is the FreeStyle Libre device, manufactured by Abbott Diabetes Care Inc. In some embodiments, a FGM does not require finger-stick calibration. And certainly one or more other types of glucose meters could be included as well.

CGM and FGM systems may measure interstitial glucose levels, while BGM systems may measure blood glucose levels. For simplicity and readability, the balance of this disclosure refers simply to a “glucose” level, or “GL.” It is understood that such references may refer to either blood glucose or interstitial glucose, as appropriate.

The connected injection device 134 is also associated with the patient 124 for medical, diabetes-treatment purposes, and is communicatively connected with the mobile device 104 via the above-described communication link 136. Device 134 may further comprise a drug or medication. In some embodiments, a system may comprise one or more devices including device 134 and a drug or medication. The term “drug” or “medication” refers to one or more therapeutic agents including but not limited to insulins, insulin analogs such as insulin lispro or insulin glargine, insulin derivatives, glucagon, glucagon analogs, glucagon derivatives, and any therapeutic agent that is capable of delivery by the above device. The drug or medication as used in the device 134 may be formulated with one or more excipients. The device is operated in a manner generally as described herein by a patient, caregiver or healthcare professional to deliver the drug to a person.

In at least one embodiment, the connected injection device 134 is or at least includes what is referred to at times in the art as a connected insulin-delivery device (e.g., a connected insulin pen, such as a pen having integrated and/or attachable electronics to auto-detect and report to another electronic device an amount of injected insulin). In various different embodiments, the connected injection device 134 takes the form of or includes one or more of the insulin-delivery devices described in any of the following patent applications, each of which is hereby incorporated herein by reference in its respective entirety:
PCT Application No. PCT/US17/65251 filed Dec. 8, 2017 and entitled Medication Delivery Device with Sensing System (Attorney Docket No. X21353);
PCT Application No. PCT/US18/19156 filed Feb. 22, 2018 and entitled Dose Detection and Drug Identification for a Medication Delivery Device (Attorney Docket No. X21457); and
U.S. Provisional Patent Application No. 62/552,659 filed Aug. 31, 2017 and entitled Dose Detection with Piezoelectric Sensing for a Medication Delivery Device (Attorney Docket No. P21462).

Moreover, in some cases, one or more of the capabilities of one of those two devices in this disclosure are also or instead covered by the other of those two devices and/or by one or more additional devices. In one example, a single device can both monitor glucose (and report back results) and inject insulin (and report back injected amounts). And certainly other combinations of capabilities could be listed here.

Furthermore, and as also described below, in some embodiments, an MMA executing on the mobile device 104 communicates for various reasons (e.g., sending dosing commands, receiving dosed-amount confirmation reports, requesting (e.g., glucose) readings, receiving one or more measured values, and/or the like) with one or more connected medical devices such as the example glucose meter 126 and connected injection device 134. Such communication may be in one-way or two-way manner with a given device. Additional examples of information that could be conveyed from a connected medical device to an MMA include error codes, device metrics, dosing records, and/or dosing confirmations. And certainly other examples could be listed here. Moreover, in some cases, direct communication links exist between various connected medical devices, such as between the glucose meter 126 and the connected injection device 134.

The conceptual-information-flow arrow 130 is meant to graphically and conceptually depict that, among other information and as is described more fully below, the HCP system 102 transmits, either directly, via data network 108, via the C3-server system 106, or via some other intermediate component or network, HCP-selected medication-dosing-regimen (e.g., bolus-dosing-regimen) prescriptions to the MMA executing on the mobile device 104. Similarly, the conceptual-information-flow arrow 132 is meant to graphically and conceptually depict that, among other information and as is described more fully below, the MMA executing on the mobile device 104 transmits, either directly or via the C3-server system 106, patient-tracked and patient-shared health data regarding the patient's diabetes and/or one or more other health-related conditions, topics, and the like.

As a general matter, it should be understood that any of the entities described herein, including but not limited to the HCP system 102, the health IT system 118, the mobile device 104 (including an MMA executing thereon), the C3-server system 106, and the administrator-portal system 148—can communicate in at least one embodiment with any other of those entities without being required to route that communication through one or more other entities. For example, in at least one embodiment, the HCP system 102 and the mobile device 104 can exchange information without that information having to pass through the C3-server system 106. In some embodiments, however, one or more entities do communicate with one another via at least one additional entity;

as an example, in at least one embodiment, data (e.g., HCP-selected-regimen data) is communicated from the HCP system 102 to the MMA executing on the mobile device 104 via the C3-server system 106.

B. Example HCP System (Example Physical Architecture)

FIG. 2 depicts an example physical architecture of the HCP system 102, in accordance with at least one embodiment. This architecture is provided by way of illustration and not limitation, as other architectures deemed suitable by those of skill in the art could be implemented. As depicted in FIG. 2, the HCP system 102 includes a communication interface 202, a processor 204, data storage 206, and a user interface 208 that includes the aforementioned display 120.

The communication interface 202 could include one or more components such as Ethernet cards and/or the like for wired communication and/or one or more components such as Wi-Fi chips and/or the like for wireless communication. The processor 204 could be a general-purpose microprocessor such as a central processing unit (CPU). The data storage 206 could be or include any suitable non-transitory computer-readable medium (CRM) such as memory (e.g., read-only memory (ROM), random-access memory (RAM)), flash memory, a solid-state drive, optical memory, and/or the like), and may contain instructions executable by the processor 204 for carrying out at least the HCP-portal-system functions described herein. The user interface 208, in addition to the display 120, may include one or more output devices such as speakers, indicator LEDs, and/or the like, and may further include one or more input devices such as a keyboard, a mouse, a trackpad, a microphone, and/or the like, as is known in the art. The various components of the HCP system 102 are depicted as communicatively interconnected via system bus 210.

Moreover, it should be understood that, in at least one embodiment of the present systems and methods, HCP system 102 runs or implements a HCP portal 572 (depicted in FIG. 5, and also referred to herein as a “HCP-portal application 572”). HCP portal 572 can be a software or firmware application executing on the hardware of HCP system 102. For example, HCP portal 572 can take the form of a widget executing within a HCP's Electronic Health Record (EHR) system. Such a widget could integrate with a HCP's EHR system using a protocol such as SMART (Substitutable Medical Apps, Reusable Technology) on FHIR (Fast Healthcare Interoperability Resources). In some embodiments, HCP portal 572 can take the form of a web application running within a web browser. In an illustrative example, an HCP logs into the HCP portal 572 by pointing a web browser on the HCP system 102 to a hyperlink, whereupon the HCP portal 572 opens in the browser. In at least one such embodiment, the HCP-portal content in the HCP portal 572 is hosted by the C3-server system 106. Thus, in some examples, the HCP system 102 stores little to no patient data (e.g., patient medical records, dosing-regimen data, and/or the like), and instead acts as a mere conduit of such patient data between its HCP user and the C3-server system 106. In some such embodiments, there is locally stored data in the form of code for the web browser itself (e.g., Microsoft Edge, Google Chrome, Apple Safari, and/or the like), as well as perhaps code for a web-application (e.g., Java) add-on. And certainly other variations could be listed here—with respect to the centralized and/or distributed nature of operable code and/or other substantive data—that those of skill in the art may deem suitable for a given implementation.

C. Example Mobile Device (Example Physical Architecture)

FIG. 3 depicts an example physical architecture of the mobile device 104, in accordance with at least one embodiment. As can be seen by inspection, FIG. 3 is similar in a number of ways to FIG. 2, and thus is not described here in as great of detail. Due to the mobile device 104 being a mobile device, however, those of skill in the art will understand that corresponding differences may be present in each of the components, such as the communication interface 302 perhaps being equipped, programmed, and configured for both wireless-wide-area-network (WWAN) communication (according to, e.g., LTE) and wireless-local-area-network (WLAN) communication (according to, e.g., Wi-Fi), as well as having additional wireless-communication capabilities such as NFC, Bluetooth, RFID, RF, and/or the like. Moreover, the processor 304 is selected in at least one embodiment to be suitable for mobile-device implementations, as is the data storage 306, which contains instructions executable by the processor 304 to carry out at least the mobile-device functions described herein, including but not limited to executing the herein-described MMA. Finally, in addition to the display 122, the user interface 308 includes one or more user-input and/or user-output components suitable for mobile-device installations (e.g., touchscreen, stylus, pointing and clicking devices, soft keys, keyboard, microphone, speaker, camera, data ports, and/or the like). The various components of the mobile device 104 are depicted as being communicatively interconnected via system bus 310.

D. Example C3-Server (Example Physical Architecture)

FIG. 4A depicts an example physical architecture of the C3 server 140, selected as an example one of the C3 servers 140-146 of the C3-server system 106, in accordance with at least one embodiment. As noted above, the C3-server system 106 could take the form of any number of servers, including just one and also including more than one. In FIG. 1, the four C3 servers 140-146 with an ellipsis between the third and fourth is intended to illustrate this numerical flexibility. As can also be seen, FIG. 4A is similar in many respects to both FIG. 2 and FIG. 3, and thus is not described herein in as great detail.

In most scenarios, the architecture of the C3 server 140 would typically be more akin to that of the HCP system 102 than that of the mobile device 104, if for no other reason than the level of performance and data processing of which such varied architectures are capable. Even in comparison with the HCP system 102, however, there are differences in the architecture of the C3 server 140 in at least one embodiment. For data-security reasons, the communication interface 402 may include only wired-communication hardware (e.g., Ethernet cards), and may include a greater number of data-communication interfaces as compared with the HCP system 102.

Similarly, the processor 404 and the data storage 406 would typically be selected so as to handle more communication, storage, data processing, and the like than would typically be asked of the HCP system 102. The data storage 406 contains instructions executable by the processor 404 for carrying out at least the C3-server functions described herein. Lastly, the user interface 408 is depicted in dashed lines to indicate that it is not present in all embodiments. For example, in some cases, the C3 server 140 is only accessible in a user-interface-like manner via data connections such as remote-desktop connections, shell connections, and/or the like. The various components of the C3 server 140 are depicted as being communicatively interconnected via system bus 410.

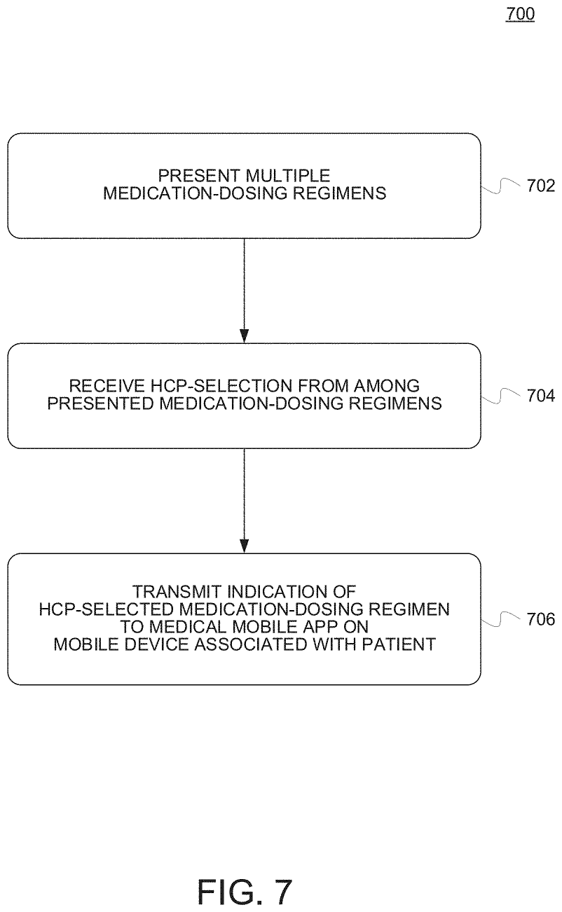

As described herein, in at least one embodiment, the C3-server system 106 collectively contains instructions that are executable by one or more of the one or more processors of the one or more C3 servers 140-146 for causing the C3-server system to carry out various combinations of the C3-server-system functions that are described herein. The below-described method 700 of FIG. 7 is one such example combination. Indeed, in at least one embodiment, the logic for implementing the C3-server system 106 is distributed across multiple physical servers. In at least one embodiment, the hardware (e.g., data storage, processor capacity, web servers, switches, routers, communication links, and the like) that are used to facilitate particular access requests and/or the like are dynamically provisioned such that different access requests may be facilitated different sets of hardware. Such hardware may be dynamically provisioned for a particular access request based on various factors such as the geographic location of the request, load balancing, required quality of service (QoS), service-agreement parameters, and/or the like. The C3-server system, then, can be thought of as a “cloud” as that term is used in the art, where that cloud includes a distributed network of servers that collectively and dynamically run virtual services. In at least one embodiment, each such virtual service is its own self-contained software process. Moreover, the particular hardware server (or servers) that actually execute a given instance of a given virtual service is dynamically allocated on the fly depending on one or more factors such as those mentioned in this paragraph.

E. Example Administrator-Portal System (Example Physical Architecture)

FIG. 4B depicts an example physical architecture of the administrator-portal system 148, in accordance with at least one embodiment. As can be seen by inspection, FIG. 4B is similar in a number of ways to each of FIG. 2, FIG. 3, and FIG. 4A, and thus is not described here in as great of detail. As stated above, some options for the administrator-portal system 148 include a desktop computer, a laptop computer, a tablet computer, a workstation, and the like. The administrator-portal system 148 can take the form of any one or more computing devices that are suitably equipped, programmed, and configured to carry out at least the administrator-portal-system functions described herein. As depicted in FIG. 4B, the administrator-portal system 148 includes a communication interface 452, a processor 454, a data storage 456 containing instructions executable by the processor 454 for carrying out at least the administrator-portal-system functions described herein, and a user interface 458, all of which are communicatively connected with one another via a system bus 460.

Moreover, it should be understood that in at least some embodiments, similar to the HCP system 102, the administrator-portal system 148 runs or implements an administrator-portal application (not shown). The administrator-portal application can be a software or firmware application executing on the hardware of the administrator-portal system 148. For example, the administrator-portal application can take the form of a web application running within a web browser. An administrator or user may log into the administrator-portal application by pointing a web browser on the administrator-portal system to a hyperlink, whereupon the administrator-portal application opens in the browser. In such embodiments, the content within the administrator-portal application may be hosted by the C3-server system 106, and little to no patient data (e.g., patient medical records, dosing-regimen data, and/or the like) may be stored locally on the administrator-portal system 148. Instead, the administrator-portal system 148 instead acts as a mere conduit of patient data between its administrator/user and the C3-server system 106.

F. Example C3-Server System (Example Functional Architecture)

FIG. 5 depicts an example functional architecture of the C3-server system 106, in accordance with at least one embodiment. It should be understood that this functional architecture is provided by way of illustration and not limitation, and that other functional architectures could be implemented as deemed suitable by those of skill in the art in various different contexts. The functional architecture of the C3-server system 106—including each of the various data stores, services, gateways, links, and interfaces—could be implemented using any combination of hardware, firmware, and/or software (wherein the firmware and/or software are run on hardware) deemed suitable by those of skill in the art. It is also noted that entities in addition to the C3-server system 106 are depicted in the architecture 500 that is shown in FIG. 5. This is clarified by the left brace labeled “106” for C3-server system 106—components outside of said left brace are associated with entities in addition to the C3-server system 106.

As can be seen in FIG. 5, the architecture 500 includes four of what are known in the computing and programming arts as “layers” separated by “interfaces”; these are logical constructs that foster, among other benefits, encapsulation and manageability of complex systems. The architecture 500 includes a data layer 502, an internal-web-services (IWS) layer 504, an externally-facing-services (EFS) layer 506, and an application layer 508. The data layer 502 is separated by a data-IWS interface 503 from the IWS layer 504, which is separated by an IWS-EFS interface 505 from the EFS layer 506, which is separated by an EFS-application interface 507 from the application layer 508. It is further noted that an interface 509 represents the logical part of the architecture 500 at which users would interact with the overall system, and in particular interact directly with the application layer 508—in other words, the interface 509 is what is often referred to in the art as the “man-machine interface.”

In the depicted embodiment, the data layer 502 includes a plurality of data stores 510a-e. Each data store 510a-e corresponds to one of the micro-services 514, 516, 518, 520, and/or 522 described herein. Data stores 510a-e collectively contain patient and/or medical data, including personally-identifiable-information (PII) such as names, addresses, telephone numbers, e-mail addresses, social security numbers, and the like. Data stores 510a-e may also collectively information other than PII, such as medical treatments, definitions, dosing regimens (i.e., templates), and the like. In at least one embodiment, the data layer 502 is implemented using the on-demand cloud computing platforms and services operated and offered by Amazon Web Services, a subsidiary of Amazon.com, Inc. of Seattle, Wash. In at least one other embodiment, the data layer 502 is implemented using the PostgreSQL product developed by Heroku of San Francisco, Calif. And certainly other options could be listed here as well. In at least one embodiment, the data layer 502 is organized according to the FHIR standards, which are put forth by Health Level Seven International (HL7) of Ann Arbor, Mich.

The IWS layer 504 includes a plurality of micro-services 514, 516, 518, 520, and 522. The ellipsis between the service 520 and the service 522 is meant to indicate that there could be any number of services in the IWS layer 504 as deemed suitable by those of skill in the art for a given implementation. Each micro-service is a data gateway implemented as a software service responsible for handling data or functionality of a certain type. Exemplary micro-services include a “person” micro-service for handling PII related to enrolled or prospective patients; an “order” micro-service for handling data related to prescription orders; an “observation” micro-service for handling data related to patient observations input by a HCP or observed by the MMA running on a mobile device; a “user access” micro-service for handling data related to users' access rights and permissions; a “client compatibility” micro-service for verifying that a user's mobile device is compatible with the functionality provided by C3-system 106, and a “substance” micro-service for handling data related to types of drugs handled or administered to/by patients. Each micro-service 514-522 has access to one data-store 510a-e via a corresponding communication link 511a-e.

Each internal service 514-522 is, in at least one embodiment, designed such that access—including read access and write access—to the data stored in the data stores 510a-e is only available—to the various gateways 524-530 of the EFS layer 506—in the manner defined and permitted by the respective internal services 514-522. In at least one embodiment, the IWS layer 504 is implemented using Amazon Web Services. In another embodiment, the IWS layer 504 is implemented using the Heroku Platform, also developed by Heroku. And certainly other options could be listed here as well.

It is further noted that the “links” that are described in connection with FIG. 5 represent logical-access links, not necessarily physical communication links; a logical-access link being present in FIG. 5 indicates that, in at least the described embodiment, proper configurations, authorizations, authentications, permissions, and/or the like have been put in place to facilitate communication between the corresponding entities.

The EFS layer 506 includes an HCP-portal gateway 524, an MMA gateway 526, an MMA gateway 528, and an admin gateway 530. Moreover, the ellipsis between the MMA gateways 526 and 528 is meant to indicate that any suitable number of MMA gateways could be implemented; the ellipsis between the mobile devices 104 and 532 in the application layer 508 indicate the same. Each MMA gateway corresponds to a specific type or version of MMA. The implementation of a dedicated MMA-gateway instance per type or version of MMA is a design choice, and others could be made. Across the IWS-EFS interface 505, it can be seen—by the system-bus-like depiction of the IWS-EFS interface 505—that any of the gateways of the EFS layer 506 are able, in this depicted embodiment, to call any of the internal services 514-522 on an as-needed basis. Each gateway 524-530 in the EFS layer 506 is designed in at least one embodiment such that access to the internal services 514-522 of the IWS layer 502 is only available to entities in the application layer 508 in the manner defined and permitted by the respective gateways 524-530. For example, in an embodiment, the HCP portal 572 running on HCP system 102 can access internal services 514-522 through HCP portal gateway 524; similarly, MMA 568 running on mobile device 104 can access internal services 514-522 through MMA gateway 526. In at least one embodiment, the EFS layer 506 is implemented using Amazon Web Services. In at least one other embodiment, the EFS layer is implemented using the Heroku Platform. And certainly other options could be listed here as well.

The applications layer 508 includes the HCP system 102 executing a Health Information Technology User Interface (HIT UI) 574, which in turn is executing a HCP portal 572. The applications layer 508 further includes the above-mentioned mobile device 104 (executing an MMA 568 in accordance with the present disclosure), a second mobile device 532 (executing another type or version of MMA, depicted as MMA 570, in accordance with the present disclosure), an admin portal 534, and an admin portal 536. As with the previously mentioned ellipses, the ellipsis between mobile device 104 and mobile device 532 is meant to indicate that any suitable number of mobile devices may be present in the application layer 508. Furthermore, a single MMA gateway (e.g., MMA gateway 526) may be connected to multiple mobile devices, each running the same type or version of MMA (e.g., MMA 568). Similarly, the ellipsis between the admin portal 534 and the admin portal 536 is meant to indicate that any suitable number of system-administrator-level portals could be implemented as deemed suitable in a given context by those of skill in the art. In an embodiment, the admin portal 534 is a clinical C3 admin server, configured for administration of the C3-server system 106 in connection with clinical aspects. In an embodiment, the admin portal 536 is a production-support C3 admin server, configured for administration of the C3-server system 106 in connection with production-support aspects. And certainly other examples could be listed here. In at least one embodiment, one or both of the admin portal 534 and the admin portal 536 are processes executed on the administrator-portal system 148. Furthermore, although no HCP-portal-related ellipsis or additional HCP portals are depicted in FIG. 5, it is also the case that any suitable number of HCP portals could be implemented in a given context.

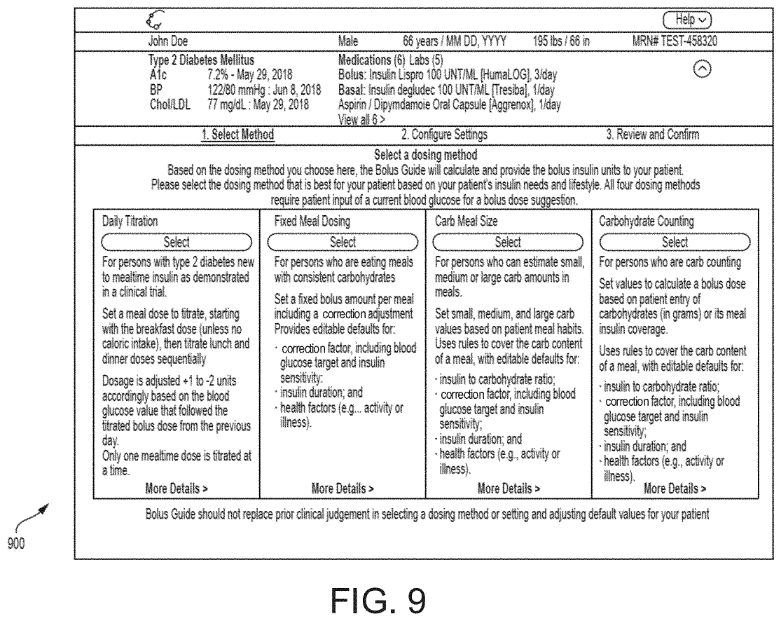

HCP system 102 can provide to a HCP a Health Information Technology User Interface (HIT UI) 574. In some embodiments, HIT UI 574 can be a web application, shell, or other suitable user-interface that allows a HCP to view, edit, create, or otherwise interact with patient or non-patient data and records stored in HIT 118. HIT UI 574 in turn can launch, at the HCP's command, HCP portal 572 as a separate user-interface within HIT UI 574—exemplary screenshots of HCP portal 572 are provided as part of FIGS. 9-18. As described previously herein, HCP portal 572 can take the form of a web application running within a web browser that allows the HCP to access or provide patient data from/to C3-server system 106. In at least some embodiments, HCP portal 572 can also interface with and access or provide patient data from/to HIT 118. In this way, a HCP can launch, with a click of a button or key, HCP portal 572 while viewing a particular patient's record within HIT UI 574 in order to prescribe a medication dosing regimen, view patient observations or logged dosing history, or access other features of the HCP portal described herein.

In some embodiments, HIT UI 574 and HCP portal 572 may both be provided by the same vendor. In other embodiments, HIT UI 574 may be provided by a first vendor, and HCP Portal 572 may be provided by a second vendor. The HCP Portal 572 may be integrated into HIT UI 574 using industry standards such as SMART on FHIR, described above. Integrating HCP portal 572 in this way allows the HCP to access, through the portal 572, patient data from HIT 118 without having to manually enter the data, and allows the HCP to select an appropriate dosing regimen without having to go back to the HIT UI 574 to review critical patient data such as medications, medical history, lab results, historical blood sugar logs, insulin dosage information, and the like. In some instances, HCP portal 572 can also be configured to pull a minimum set of demographic data pertaining to a particular patient (e.g., patient name, date of birth, unique identifier for the patient, or unique identifier for the patient's EHR) from HIT 118, and display this demographic data in a banner or other prominently displayed region. This can help ensure that the HCP is working in the correct patient record.

Although HCP portal 572 is depicted as being encapsulated within HIT UI 574, HCP portal 572 may also be implemented as a standalone application outside of any Health IT system. In such cases, the HCP system 102 would execute or implement HCP-Portal 572 without any HIT UI 574. In such embodiments, HCP portal 572 may optionally still draw patient data from a Health IT system, but may not launch as a separate window or graphical user-interface from within the user-interface of a Health IT system. Of course, HCP portal 572 may also be configured to not communicate with any Health IT system at all—in such embodiments, patient medical and biographical data may need to be manually entered and stored into HCP portal 572.

Across the EFS-application interface 507, the HCP system 102 alone has access, via the link 558, to the HCP-portal gateway 524; the MMA 568 (on the mobile device 104, or on multiple mobile devices) alone has access, via the link 560, to the MMA gateway 526; the MMA 570 (on the mobile device 532, or on multiple mobile devices) alone, via the link 562, has access to the MMA gateway 528; and the C3 admin gateway 530 is accessible by both the C3 admin portal 534 (via the link 564) and the C3 admin portal 536 (via the link 566). Such restricted access is, in the depicted embodiment, two-way, in that the C3-server system 106 can only access the HCP system 102 via the HCP-portal gateway 524 and the link 558; the C3-server system 106 can only access the MMA 568 via the MMA gateway 526 and the link 560, and similarly with the MMA 570 via the MMA gateway 528 and the link 562, as well as the C3 admin gateway 530 and the links 564 and 566 to the respective C3 admin portals 534 and 536.

G. Example Ecosystem

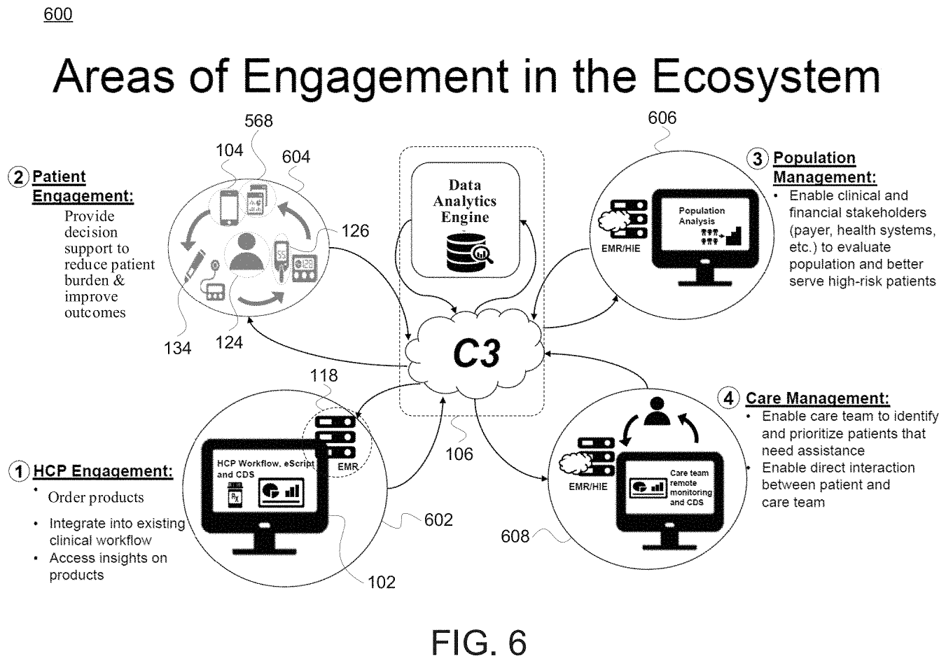

FIG. 6 depicts various entities in an example communication-and-processing ecosystem 600, in accordance with at least one embodiment. Of course the term ecosystem is not used here in the biology-related sense of the word, but rather in a communication-and-processing computing context. Where appropriate, elements that are discussed above in connection with FIGS. 1-5 are also labeled in FIG. 6 in order to assist the reader's understanding by placing such elements in the context of the ecosystem 600, which includes an HCP-engagement area 602, a patient-engagement area 604, a population-management area 606, and a care-management area 608, all of which communicatively interact and interrelate with one another via the C3-server system 106, which is shown in FIG. 6 as including a data-analytics engine in this depiction. As mentioned above, it is also contemplated that the C3-server system 106 in some embodiments communicatively connects (e.g., shares data with (perhaps only conditionally upon patient opt-in) and receives data from) payer entities such as insurance companies, Medicare, and the like.

The data analytics engine may be configured to analyze data collected by or accessible through patients' mobile devices and/or health record data to drive insights regarding patient behavior or symptoms related to a disease or condition. The analyzed data may be unique to a single patient, and used to derive insights specific to that patient. For instance, the data analytics engine may determine, through analyzing a diabetic patient's log of glucose readings, that the patient tends to exhibit hyperglycemia or hypoglycemia under certain conditions, e.g., at certain times of day, after eating certain types of meals, on certain days of the week, when located at or en route to/from certain geographic locations, before/during/after certain types of physical activity, and other types of conditions. Based on these insights, the data analytics engine may take a variety of actions, such as notifying the patient's HCP, pushing educational content to the patient related to the observed hyper- or hypoglycemic episodes, or prompting the patient to change his/her behavior at specific times or locations when or where they are likely to have the most effect (e.g., prompting the patient to take medication just before bedtime, immediately after exercising, or when they are at or en route to/from a particular geographic location).

The HCP-engagement area 602 of the ecosystem 600 includes the HCP system 102 and the health IT system 118, and as stated in FIG. 6 enables HCPs to order products from the company or other organization that operates the C3-server system 106, access insights into such products, and integrate the present systems and methods into their existing clinical workflow.

The patient-engagement area 604 of the ecosystem 600 includes the mobile device 104 executing the MMA 568, the patient 124, the glucose meter 126, the connected injection device 134, and a number of other patient-related medical devices such as a blood-pressure or other meter and the like. In the patient-engagement area 604 of the ecosystem 600, the goals of the present systems and methods include, as stated in FIG. 6, to provide relevant and valuable decision support to reduce patient burden and improve outcomes.

The population-management area 606 of the ecosystem 600 includes computing equipment (perhaps similar in physical architecture to that of the HCP system 102) as well as an EMR/HIE (Health Information Exchange) system for proper storage and maintenance of patients' personal medical information. In the population-management area 606 of the ecosystem 600, the goals of the present systems and methods include, as stated in FIG. 6, to enable clinical and financial stakeholders (payers, health systems, etc.) to evaluate populations and better serve high-risk patients.

The care-management area 608 of the ecosystem 600 also includes computing equipment similar to that of the HCP system 102, as well as its own EMR/HIE system, and is generally dedicated to remote monitoring and clinical decision support (CDS) involving what is often referred to as “the care team” for a given patient. This care team may include multiple different HCPs (different types of doctors, different types of nurses, therapists, caretakers, etc.) having various different roles that all pertain to health and well-being of the patient. In the care-management area 608 of the ecosystem 600, the goals of the present systems and methods include, as stated in FIG. 6, to enable the care team to identify and prioritize patients that need assistance and also to enable direct interaction between the patient and the care team.

In at least one embodiment, a companion MMA is provided that allows caregivers, loved ones, and/or the like access to some or all of the data received, recorded, tracked, or entered by the patient 124 via the MMA 568. In at least one such embodiment, a person using the companion MMA is able to view a patient's logbook (including, e.g., glucose measurements, dosage information, meal-related information, and/or the like), as well as prescribed dosing regimen(s), and/or any other type of data deemed suitable by those of skill in the art for a given implementation. The amount and/or types of data viewable via the companion MMA could be set to default values and also could be adjustable by an HCP, by the patient 124, and/or by one or more other individuals, depending on the design parameters of a given implementation.

In accordance with the present systems and methods, the C3-server system 106 provides and supports a number of features that further the above-mentioned goals and others in the ecosystem 600. One such feature is data storage, in that the C3-server system 106 in at least one embodiment securely stores data, which allows the HCP system 102 and the MMA 568 to securely communicate with one another. The C3-server system 106 further enables MMA users to securely back up their medical data and to achieve data consistency across multiple devices (e.g., in embodiments in which the MMA app 568 is instantiated on the mobile device 104 as well as perhaps another mobile device associated with the patient 124, who may have both a smartphone and a tablet, as is quite common, and/or in embodiments in which the patient 124 has access to similar functionality and information via, e.g., a web portal, as is also commonly implemented in the art).

In various embodiments, as described herein, an HCP, via the HCP system 102 and the C3-server system 106, sets up dosing regimens and sends these to the MMA 568 via the secure-data-transfer capabilities of the C3-server system 106. Furthermore, in various embodiments, the C3 server makes patient historical data, dosing-regimen status, and the like available via the HCP system 102 and/or the MMA 568. Moreover, in at least one embodiment, secure communication (i.e., messaging or inbox functionality) is facilitated by the C3-server system 106 between the HCP via the HCP system 102 and the patient 124 via the MMA 568 and the mobile device 104. Furthermore, in various different embodiments, the C3-server system 106 facilitates software updates, recall notifications, and/or other functions deemed suitable by those of skill in the art for a given implementation or in a given context.

### I. Example Operation

A. HCP Portal

FIG. 7 depicts an example method 700, which may be carried out by the C3-server system 106, in accordance with at least one embodiment. It should be understood that the description herein of the C3-server system 106 carrying out the method 700 is provided for illustration and not by way of limitation, and that the method 700 could be carried out by any device or set of devices that is suitably equipped, programmed, and configured to do so.

At step 702, the C3-server system 106 presents via the HCP portal 572, utilizing the user interface 208 of the HCP system 102, multiple medication-dosing regimens pertaining to the patient 124. In at least one embodiment, each of the presented medication-dosing regimens is associated with a respective different medication-dosing algorithm. Exemplary medication-dosing regimens and algorithms are discussed in greater detail below.

Any one or more of the presented medication-dosing regimens could be what are known in the art as an open-loop regimen or a closed-loop regimen. Generally stated, a closed-loop regimen iteratively adjusts doses (as to, e.g., amount, timing, and/or the like) of one or more medications in an automated manner based at least in part on automatically sensed feedback such as measurements (of, e.g., glucose level) taken with respect to the patient, while an open-loop regimen does not iteratively adjust doses of medication in an automated manner. It is often the case, however, that open-loop medication-dosing regimens account for similar historical data in more of a manual manner, in which the patient manually inputs feedback parameters. Open-loop medication-dosing regimens may also require the patient to manually control dispensing of medication. More generally, the present systems and methods are not limited to any particular type of dosing regimen, to any particular medication or type of medication, or to any particular manner of delivery (e.g., injection, intravenous, by mouth, etc.) of the medication to the patient.

At step 704, the C3-server system 106 receives, via HCP portal 572 utilizing the user interface 208 of the HCP system 102, an HCP selection (for the patient 124) of at least one of the presented medication-dosing regimens. In the parlance of this disclosure, the selected at least one regimen is referred to as “the at least one HCP-selected medication-dosing regimen,” “the at least one HCP-selected regimen,” and/or the like.

In at least one embodiment, the medication-dosing regimens presented in step 702 include one or more insulin-dosing regimens, and the at least one HCP-selected medication-dosing regimen includes at least one HCP-selected insulin-dosing regimen. Any number of the presented regimens could be basal-insulin-dosing regimens and any number could be bolus-insulin-dosing regimens. In some cases, either a basal-insulin-dosing regimen or a bolus-insulin-dosing regimen is selected by the HCP for the patient 124. In some cases, an HCP simultaneously prescribes both one or more basal-insulin-dosing regimens and one or more bolus-insulin-dosing regimens to the patient 124.

In at least one embodiment, the at least one HCP-selected bolus-insulin-dosing regimen includes a type referred to herein as a daily-titration bolus-insulin dosing regimen that is associated with a type of dosing algorithm that is referred to herein as a daily-titration bolus-insulin-dosing algorithm. Other types of HCP-selected bolus-insulin-dosing regimens include a type referred to herein as a fixed-dose bolus-insulin dosing regimen that is associated with a type of dosing algorithm that is referred to herein as a fixed-dose bolus-insulin-dosing algorithm; a type referred to herein as a carbohydrate-bolus-calculator bolus-insulin dosing regimen that is associated with a type of dosing algorithm that is referred to herein as a carbohydrate-bolus-calculator bolus-insulin-dosing algorithm; and a type referred to herein as a carbohydrate-meal-size-based-calculator bolus-insulin dosing regimen that is associated with a type of dosing algorithm that is referred to herein as a carbohydrate-meal-size-based-calculator bolus-insulin-dosing algorithm. Examples of each of those above-mentioned four types of open-loop bolus-insulin dosing regimens/algorithms is described below in the correspondingly titled subsection.

At step 706, the C3-server system 106 transmits, to the MMA 568 on the mobile device 104 associated with the patient 124, HCP-selected-regimen data that is indicative of the at least one HCP-selected regimen of step 704.

Although example method 700 has been described above as being implemented by C3-server system 106, method 700 may also be implemented directly by HCP system 102. In such embodiments, HCP portal 572 may not be a web application hosted by C3-server system 106 over a network connection—instead, HCP portal 572 may comprise a user-interface locally hosted and executed by HCP system 102. In these cases, HCP system 102 presents multiple medication-dosing regimens to the HCP (step 702), receives a HCP-selection from among presented medication-dosing regimens (step 704), and transmits the HCP-selected-regimen data to the MMA 568, all without any assistance from or communication with a C3-server system 106. In such embodiments, HCP system 102 can communicate directly with mobile device 104 via data network 108, without requiring that data be passed through or processed by C3-server system 106.

Regardless of whether method 700 is implemented by C3-server system or by HCP-portal 102, in at least some embodiments the MMA 568 is configured to provide, via the user interface 308, dosing recommendations based on the at least one HCP-selected regimen. In some embodiments, the MMA 568 presents a prompt via the user interface 308 providing the options for the patient 124 to accept or reject the recommended (i.e., prescribed) dosing regimen. In at least one embodiment, the C3-server system 106 is configured to disable the MMA 568 in response to receiving a regimen-rejected indication indicating that the MMA 568 had received, via the user interface 308, a patient-rejection indication associated with the at least one HCP-selected regimen. Disabling the MMA 568 may comprise preventing the user from accessing some or all of the functionality of the MMA 568, or preventing the user from accessing some or all of the data stored in the MMA 568. For example, disabling the MMA 568 may comprise preventing the user from accessing or using the dosing regimen prescribed or recommended by the HCP. In other cases, the C3-server system 106 receives instead a regimen-accepted indication indicating that the MMA 568 had received, via the user interface 308, a patient-acceptance indication associated with the at least one HCP-selected regimen.

In at least one embodiment, it is the receipt by the MMA 568 of the HCP-selected-regimen data of step 706 that unlocks the at least one HCP-selected regimen on the MMA 568. In some cases, this unlocking includes the MMA 568 being enabled to access implementation data—already stored on the mobile device 104 (potentially as part of the MMA 568)—for the at least one HCP-selected regimen only after receipt of the HCP-selected-regimen data. In other implementations, this unlocking includes the MMA 568 transitioning from inoperable to operable to download implementation data for the at least one HCP-selected regimen only after receipt of the HCP-selected-regimen data. In these implementations, implementation data for the at least one HCP-selected regimen may be downloaded from the C3-server system 106 and/or the HCP system 102.

In some cases, this unlocking only occurs responsive to the MMA 568 receiving via the user interface 308 a patient-acceptance indication with respect to the at least one HCP-selected regimen. Allowing the MMA 568 to unlock or download a particular HCP-selected regimen only after receipt of the HCP-selected-regimen data can be useful for ensuring compliance with regulations enforced by the United States Food and Drug Administration (FDA). Such FDA regulations may require that certain dosing regimens be accessible by a patient only with a prescription from a qualified and licensed physician. By restricting access to a dosing regimen until receipt of the HCP-selected-regimen data, the MMA 568 in those embodiments ensures that patients cannot access a dosing regimen without a required prescription.

Embodiments that store implementation data for all regimens presented to and selectable by the HCP at steps 702 and 704 on the mobile device 104 (e.g., as part of the MMA 568), and unlock one or more regimens on the mobile device only after receipt of the HCP-selected-regimen data transmitted in step 706, confer several technical advantages that improve the efficiency and/or functionality of the present systems and methods. For example, storing implementation data for all regimens on mobile device 104 allows an HCP to enable a particular prescribed regimen on a patient's mobile device 104 without requiring that the mobile device 104 download additional, potentially sizable, implementation data for the prescribed regimen. This allows the patient to more easily access and implement the prescribed regimen if the patient's mobile device 104 only has an intermittent or low-bandwidth communication link 112 with data network 108 (e.g., in the event of a network failure, or if the mobile device is put in airplane mode).

Embodiments that involve bundling implementation data for all regimens as part of the MMA 568 also allow the MMA 568 to be posted for easy access by patients on certain popular online repositories for mobile applications. Such online mobile-application repositories may include repositories managed by third-party entities other than the entity (or entities) that owns, maintains, and/or is affiliated with the HCP system 102, the administrator portal 148, or the patient 124; such online mobile-application repositories can include the App Store maintained by Apple, Inc., or Google Play maintained by Google LLC. Such online repositories typically require that posted applications include all implementation logic at the time of posting. In other words, to ensure the security and stability of applications running on mobile devices, administrators of such online repositories may forbid application developers from updating or changing the code of posted applications unless each update is submitted, reviewed and/or approved by the third-party entity (e.g., Apple or Google) that maintains the online repository. As such, embodiments that involve downloading implementation data to the mobile device 104 each time a new dosing regimen or algorithm is prescribed may require a time-consuming submission, review, and/or approval process by the third-party entity. This is a problem that is unique to and specifically arises in the realm of medical mobile applications that enable access to dosing regimens that, per FDA regulations, should be accessible only to patients with a prescription. By bundling implementation data for all regimens as part of the MMA 568 at the time of posting, and selectively unlocking a prescribed regimen only upon receipt of HCP-selected-regimen data, the MMA 568 avoids the need for such a time-consuming submission, review, and/or approval process each time a patient is prescribed a new dosing regimen. This bundling also increases the security and stability of the MMA.

As described above, in at least one embodiment, the MMA 568 is communicatively connected via its host mobile device 104 with one or more insulin-delivery and/or insulin-monitoring devices associated with the patient 124. The glucose meter 126 is an example of such an insulin-monitoring device. In at least some embodiments in which the MMA 568 is communicatively connected with an insulin-delivery device associated with the patient 124, the MMA 568 communicates with that insulin-delivery device to carry out the at least one HCP-selected regimen. The connected injection device 134 is an example of such an insulin-delivery device. In some embodiments, the C3-server system 106 and/or the HCP system 102 receive patient data—examples of which (such as insulin-injection-record data and glucose-measurement data) are described herein—relayed by the MMA 568 from the insulin-delivery-and/or-monitoring device.

B. MMA on Mobile Device

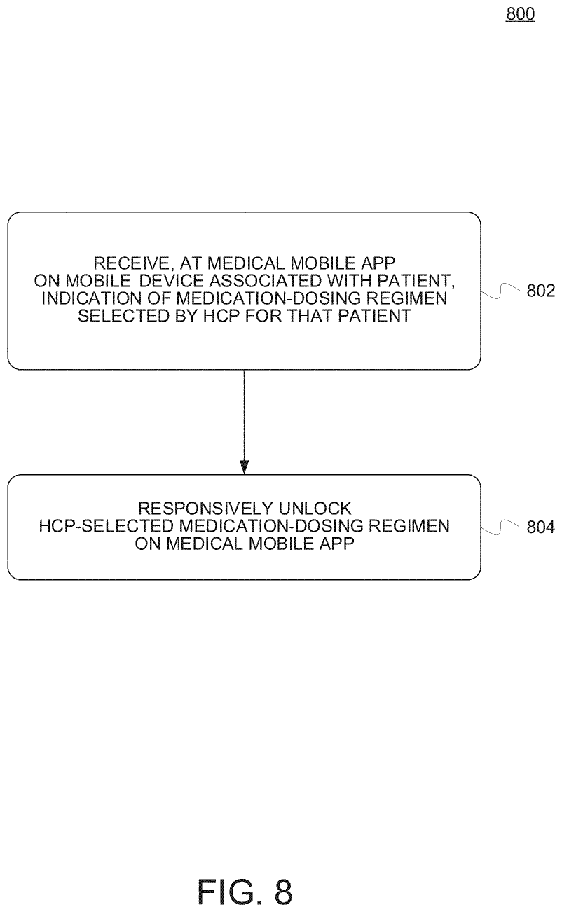

FIG. 8 depicts an example method 800, which may be carried out by the mobile device 104 executing the MMA 568, in accordance with at least one embodiment. It should be understood that the description herein of the method 800 as being carried out by the MMA 568 executing on the mobile device 104 is provided for illustration and not by way of limitation, and that the method 800 could be carried out by any device or set of devices that is suitably equipped, programmed, and configured to do so.

At step 802, the MMA 568 receives HCP-selected-regimen data that is indicative of an HCP selection of at least one medication-dosing regimen from among a plurality of medication-dosing regimens, each of which is associated with a respective different medication-dosing algorithm. In at least one embodiment, the HCP selection was made for the patient 124 via the HCP system 102 (e.g., via a web interface (HCP portal 572) provided by the C3-server system 106 and accessed by the HCP via the HCP system 102). As described above, the MMA 568 could receive the HCP-selected-regimen data directly from the HCP system 102 or, as described in this example, from the C3-server system 106. The at least one HCP-selected medication-dosing regimen could include any of the possible regimens or regimen combinations described herein.

At step 804, the MMA 568 unlocks the indicated HCP-selected medication-dosing regimen on the MMA 568 responsive at least in part to having received the HCP-selected-regimen data at step 802. As discussed previously, in at least one embodiment, step 804 involves accessing previously inaccessible implementation data for the HCP-selected-regimen that was pre-stored on the mobile device 104. These embodiments can be desirable for at least the technical reasons discussed above. In some embodiments, step 804 involves downloading previously inaccessible implementation data for the HCP-selected-regimen.

In at least one embodiment, subsequent to carrying out step 802 and prior to carrying out step 804, the MMA 568 presents via the user interface 308 of the mobile device 104 a prompt for patient acceptance or patient rejection of the at least one HCP-selected medication-dosing regimen, and then receives a user input via the user interface 308 corresponding to the presented prompt; in at least one such embodiment, the MMA 568 carrying out step 804 is conditioned upon the received user input being a patient-acceptance indication with respect to the at least one HCP-selected medication-dosing regimen. Moreover, in at least one embodiment, the MMA 568 transmits the received user input to the C3-server system 106 and/or the HCP system 102.

In at least one embodiment in which the prescribed regimen is or includes a bolus-insulin-dosing regimen, once a patient-acceptance indication has been received, indicating that the user has accepted a dosing-regimen prescription, the MMA 568 presents the user with an option to enter a bolus-dose-recommendation workflow, during which, in at least one embodiment, the MMA 568 prompts the user to select a meal time, prompts the user to enter and/or confirm their glucose measurement, and accordingly presents to the user a bolus-dose recommendation. The user may indicate to the MMA 568 that he/she has taken the recommended dose, in which case the MMA 568 will make a record in memory that the recommended dose. Alternatively, the user may indicate to the MMA 568 that he/she has taken a dose of insulin that differs from the recommended dose, and the MMA 568 will record the amount of insulin actually taken. The record may include the time at which the dose was taken, which may be automatically set at the time when the user's confirmation was received, or at a time indicated by the user.

In at least one embodiment, the MMA 568 via the mobile device 104 is communicatively connected with an insulin-delivery (and/or glucose monitoring) device associated with the patient; in at least one such embodiment, the MMA 568 communicates with the insulin-delivery device to carry out the HCP-selected regimen, or to receive information regarding doses manually delivered by the patient using the insulin-delivery device. In at least one embodiment, the MMA 568 receives patient data from the insulin-delivery device and relays the received patient data to the C3-server system 106 and/or the HCP system 102. The patient data could include insulin-injection-record data (e.g., including the times and amounts of insulin delivered in previous doses) and/or glucose-measurement data, among numerous other examples that could be listed here. In at least one embodiment, the MMA 568 provides, via the user interface 308 of the mobile device 104, dosing recommendations based on the HCP-selected regimen.

In at least one embodiment, the MMA 568 may include a safety feature that prompts the patient if it detects that the patient may be taking an unsafe or incorrect dose. For example, the MMA 568 may calculate an expected “baseline” amount of medication expected for each dose. This “baseline” dosage may be determined in multiple ways. For example, the “baseline” dosage may be a pre-set amount of medication programmed by the HCP and/or the patient, or it may be derived algorithmically and automatically from the patient's medical or biographical information (e.g., gender, age, weight, type and severity of disease, and the like). The “baseline” dosage may also be derived from a sliding mean, mode, or median average of a certain number of previous doses (e.g., the past three, five, ten, or more doses), or of the doses taken in a previous sliding window of time (e.g., in the past three, five, ten, or more days). In some embodiments, the “baseline” dosage may vary based on time-of-day, or on rising- or falling-trends detected in previous doses. Before recommending a dose based on the HCP-selected regimen, the MMA 568 may compare the recommended dose with this “baseline” dosage. If the MMA 568 detects that it is recommending a dose that differs significantly from this baseline dosage, such as if the recommended dose differs from the baseline by a certain absolute amount of medication, or by a certain percentage, the MMA 568 may warn the patient and prompt him/her to confirm the dose. It may be, for instance, that the recommended dose is based on incorrectly entered patient inputs, such as incorrectly entered insulin-on-board parameters, meal size values, or current glucose levels. Alternatively or in addition, if the user indicates to the MMA that he/she plans to take an amount of medication different from the recommended amount, the MMA 568 may similarly determine whether the user-indicated amount differs significantly from the baseline dosage. If the user-indicated amount differs significantly, the MMA 568 may warn the patient that the indicated dosage is significantly higher- or lower-than the baseline dosage. This extra prompt and warning may help warn the user that he or she may be about to take an amount of medication that is unsafe or incorrect. If appropriate, the user may override the warning.

In at least one embodiment, the MMA 568 provides at least the following two levels of access: general access, available to anyone to download, self-register, and use; and prescription access, which provides additional functionality that is unlocked through an electronic dosing regimen prescription. In an embodiment, general access offers features including (i) logging diabetes-related data, which could include glucose level (GL), basal insulin, bolus insulin, carb input, and exercise/stress/illness factors, as examples and (ii) accessing visualizations (e.g., graphs) of user data, while in an embodiment, prescription access unlocks bolus insulin dose recommendations through the implementation of a dosing regimen. In some embodiments, the appearance of the MMA 568's user interface changes depending on whether the MMA 568 is in general access mode or prescription access mode (and/or perhaps other modes). Such changes could include but are not limited to additional and/or fewer menu items, color-scheme changes, icon changes, layout changes, and/or the like.

In at least some embodiments, the general access (non-prescription) mode of MMA 568 provides one or more of the following features:
Data Entry
   The user can enter diabetes-related data to facilitate tracking and viewing of data by the HCP.
   The user is able to enter bolus insulin doses, basal insulin doses, glucose readings (manually or through wireless connection to glucose meter), and carbohydrate intake.
   The user can also log the following modifiers, if applicable, and yes/no data points: activity, stress, illness, steroids, and menstruation.
   The patient can input values into their MMA such as glucose values (either auto from sensor or manual), current activity level, current food eaten, carb content of food, physiological characteristics such as body weight, etc.
Digital Logbook
   The user can track diabetes-related data so that all of their data is easily accessible and viewable in one location.
   The user can view any of their entered data in a digital logbook format.
Glucose-Meter Pairing
   Simplifies the glucose entry process for users who want to store all of their data in one location or want to use glucose data to receive a dose recommendation.
   The user can select the option to pair a device, such as a blood glucose meter, a continuous-glucose meter, flash-glucose monitor, or other glucose monitoring device. Once they have paired their glucose monitoring device, the MMA will display their most recent glucose measurement when the user navigates to a bolus-dose-recommendation workflow (available in prescription mode only) or a data-entry workflow (available in general access mode).
   Users can pair with connected glucose meters that claim compliance to the Continua standard (a.k.a. the Continua Design Guidelines), published by Personal Connected Health Alliance (PCHAlliance) of Arlington, Va.
   In some embodiments, users can conduct experiments by doing activities and measuring the effect on their glucose level. Users can review experiments that they have done. Such experiments could be based on activity, food, and/or other factors. In some embodiments, third-party data sources (e.g., HealthKit, FitBit, and/or the like) create experiments for users. Indeed, in some embodiments, the MMA can connect and communicate with wearables such as the FitBit, the Apple Watch, and/or the like) to facilitate monitoring and/or integration of insights regarding one or more physiological parameters.
Data Visualizations
   The user can view their collected data over a range of time periods to make the data more understandable.
   The user can navigate to the section of the MMA that hosts data visualizations. They can then select the type of data and time period that they wish to view.
   The MMA will also show a pie chart of recorded glucose values to show the number above range, in range, below range, and those falling into hypoglycemia levels (<70 mg/dL).
Reminders
   Helps the user to remember to test their glucose or enter the bolus dose recommendation workflow when their schedule is busy.
   The user can set reminders for times that they would like to test their glucose level and/or administer insulin.
   The user will receive a message on their mobile device with the reminder.
Onboarding
   The user can register the MMA and learn about useful functions.
   The user can register to use the MMA by creating login credentials, entering basic information about themselves, and accepting the terms of use.
   The MMA will offer optional activities (e.g., tutorials) to help users learn about useful functions, including pairing a glucose meter or setting personalized reminders.
Education on Diabetes
   Helps the user learn about their diabetes from reputable sources.
   The user can access a link to diabetes education from within the MMA. Such a link could launch within the MMA or launch a browser application, among other possibilities.

C. Example HCP-Portal Screenshots

As described below, FIGS. 9-21 depict various screenshots of various different embodiments of the HCP system 102.

FIG. 9 is a screenshot 900 in which an HCP user is operating in a “Select [Dosing] Method” tab, and in which four bolus-insulin dosing regimens are listed as options for the HCP to select—these four bolus-insulin dosing regimens are described in further detail below. In an embodiment, clicking on any one of the presented regimens presents a pop-up window that provides further detail. In at least one embodiment, when an HCP selects one of these four dosing regimens, the HCP system 102 sends HCP-selected-regimen data to the mobile device 104, which enables the patient to access the selected dosing regimen on the mobile device.

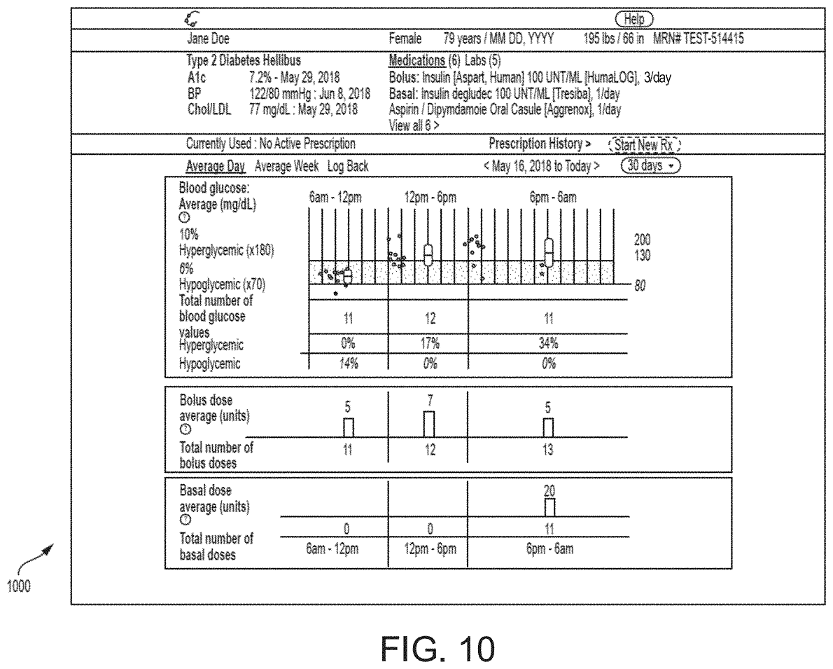

FIG. 10 is a screenshot 1000 showing electronic health record (EHR) data pertaining to one exemplary patient, depicting various patient data in graphical form. In this screenshot 1000, the exemplary patient has not yet been prescribed a dosing regimen.

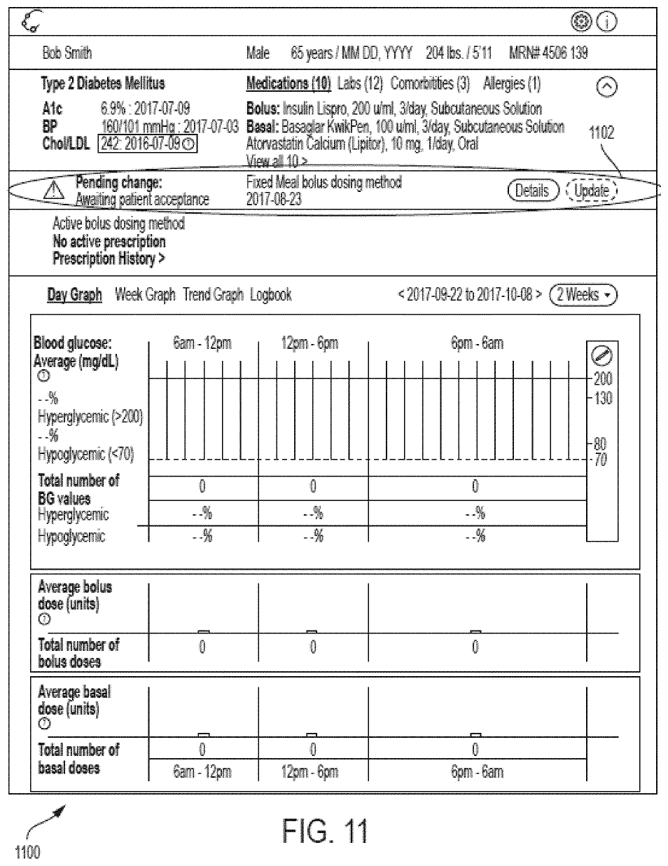

FIG. 11 is a screenshot 1100 in which an alert message 1102 is being displayed indicating that a pending change to a dosing recommendation or prescription is still awaiting patient acceptance. In this screenshot 1100, the patient has no active dosing methods prescribed, and has no historical recorded data regarding glucose values or dosage information. The patient has been prescribed a fixed meal bolus dosing method, but the patient has not yet accepted the prescribed dosing method.

FIG. 12 is a screen shot 1200 in which an alert message 1202 is being displayed regarding a pending change to a dosing recommendation or prescription is still awaiting patient acceptance. In this screenshot 1200, the patient had previously been prescribed the carbohydrate-bolus-calculator bolus-insulin dosing regimen, but has now been prescribed the fixed-dose bolus-insulin-dosing regimen. The patient has not yet accepted the newly prescribed dosing method.

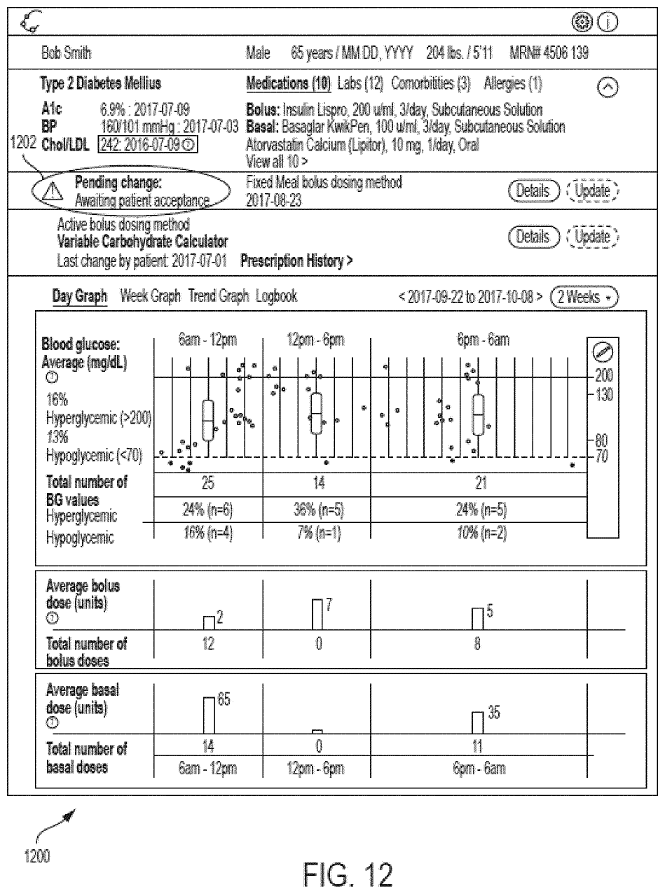

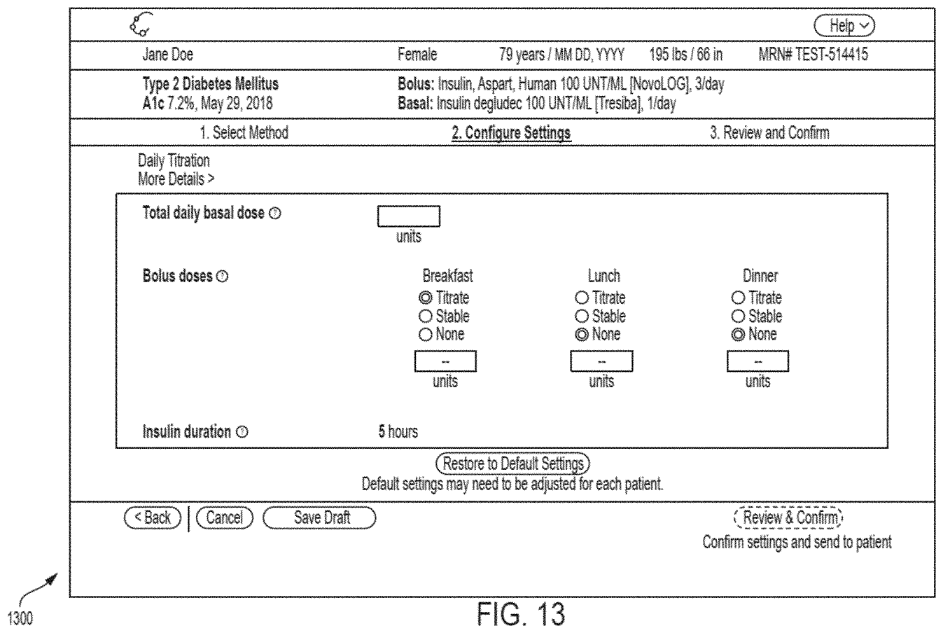

FIG. 13 is a screenshot 1300 depicting a “Configure Settings” tab where the selected dosing regimen is the daily-titration bolus-insulin-dosing regimen, also referred to simply as the “Daily Titration” regimen. As shown in FIG. 13, the daily titration regimen settings tab gives the HCP the option to prescribe bolus dose options for three meals: breakfast, lunch, and dinner. For each meal, the HCP can choose between “titrate”, “stable,” and “none.” The titrate option causes the dose recommended for that meal to be titrated according to the algorithm described herein below. The “stable” option allows the HCP to specify a fixed bolus dose for that mealtime. The “none” option means no bolus dose is prescribed for that time.

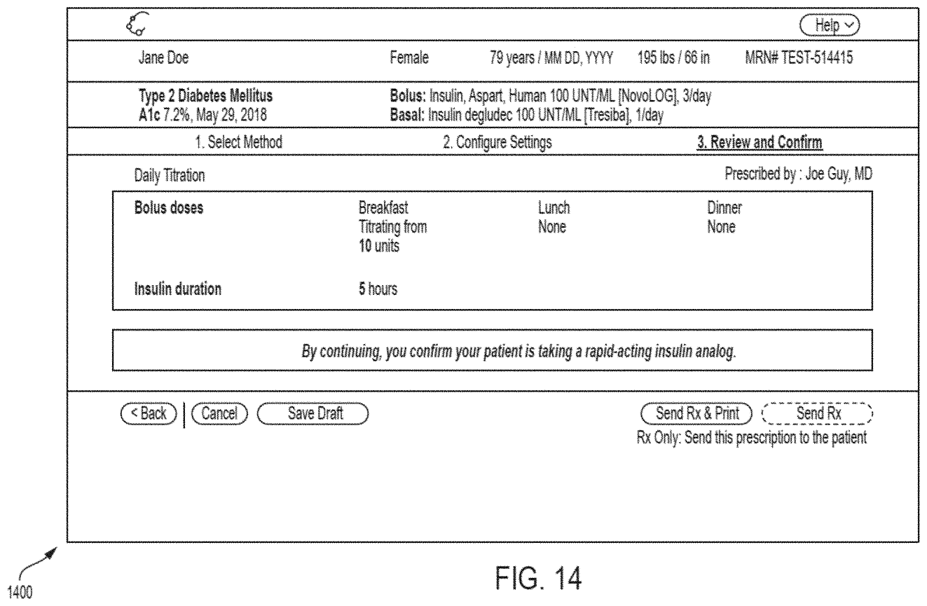

FIG. 14 is a screenshot 1400 depicting a “Review and Confirm” tab where again the selected dosing regimen is “Daily Titration.” Here, the HCP can review and confirm a prospective Daily Titration dosing regimen before prescribing it to the patient.

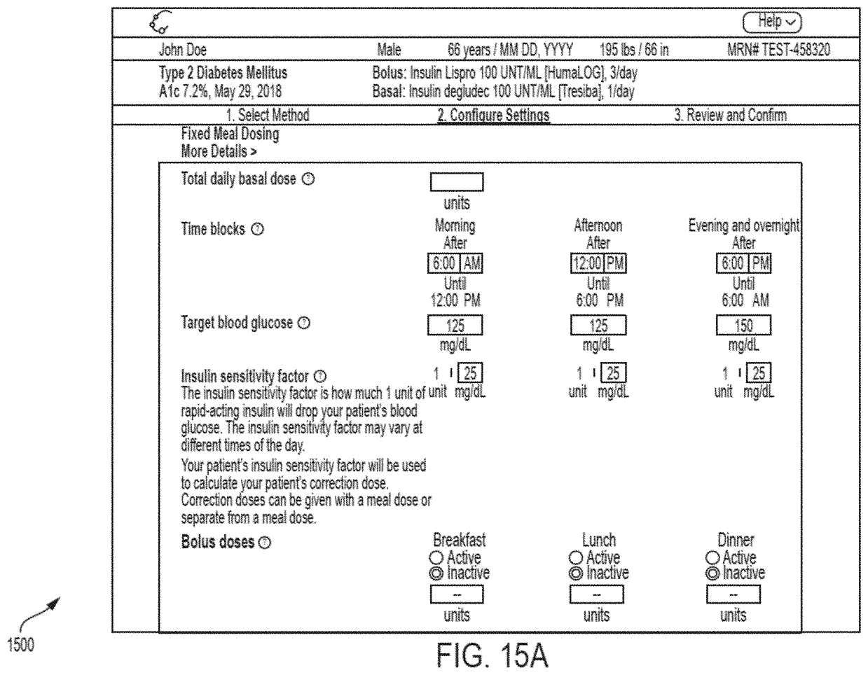

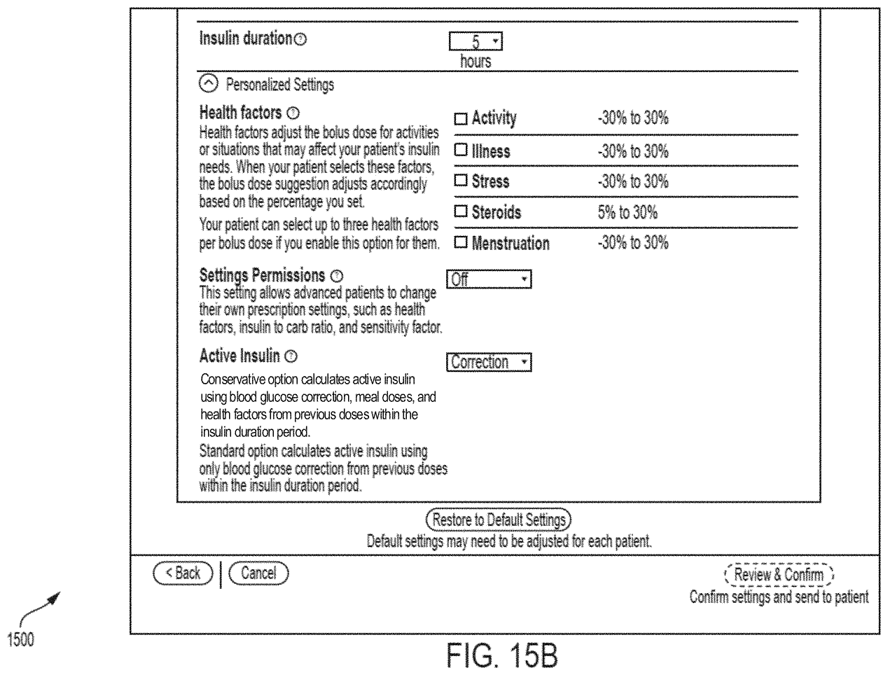

FIGS. 15A and 15B is a screenshot 1500 depicting a “Configure Settings” tab where the selected dosing regimen is the fixed-dose bolus-insulin-dosing regimen, also referred to simply as the “Fixed Meal Dosing” regimen. The HCP user is presented with an opportunity to confirm the customizable details of the regimen for the particular patient in question. For example, the HCP user can customize bolus dosage settings for three time blocks: (i) morning, (ii) afternoon, and (iii) evening and overnight. The beginning time of each time block can be adjusted by the HCP. For each time block, the HCP user can select a target glucose level, an insulin sensitivity factor (described in further detail below), a bolus dose, options to enable various health factors (e.g., activity, illness, stress, steroids, and menstruation, described in further detail below), settings permission (which, if enabled, allows the patient to change their own prescription settings), and active insulin (described in further detail below).

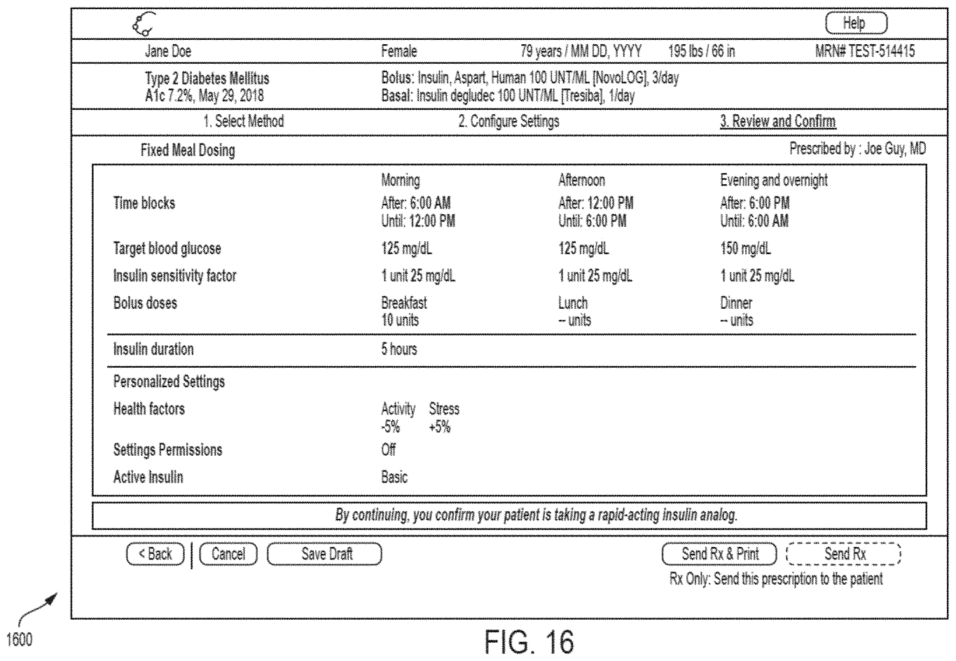

FIG. 16 is a screenshot 1600 depicting a “Review and Confirm” tab where again the selected dosing regimen is the Fixed Meal Dosing regimen. Here, the HCP can review and confirm a prospective Fixed Meal Dosing regimen before prescribing it to the patient.

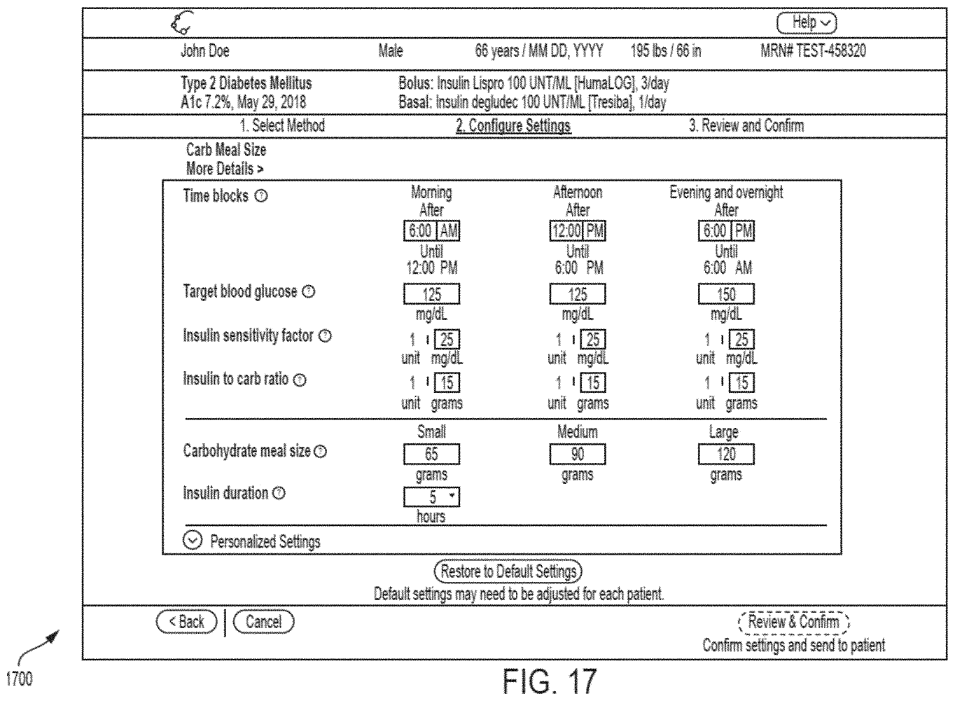

FIG. 17 is a screenshot 1700 depicting a “Configure Settings” tab where the selected dosing regimen is the carbohydrate-meal-sized-based-calculator bolus-insulin dosing regimen, also referred to simply as the “Carb Meal Size” regimen. As shown in FIG. 17, the carb meal size regimen settings tab gives the HCP the option to prescribe bolus dose options for three time blocks: (i) morning, (ii) afternoon, and (iii) evening and overnight. The beginning time of each time block can be adjusted by the HCP. For each time block, the HCP user can select a target glucose level, an insulin sensitivity factor (described in further detail below), and an insulin-to-carb ratio (described in further detail below). Independent of the time periods, the HCP user can also select an expected number of grams of carbohydrates from each of a “small”, “medium”, and “large” meal. Finally, the HCP user can select an insulin duration.

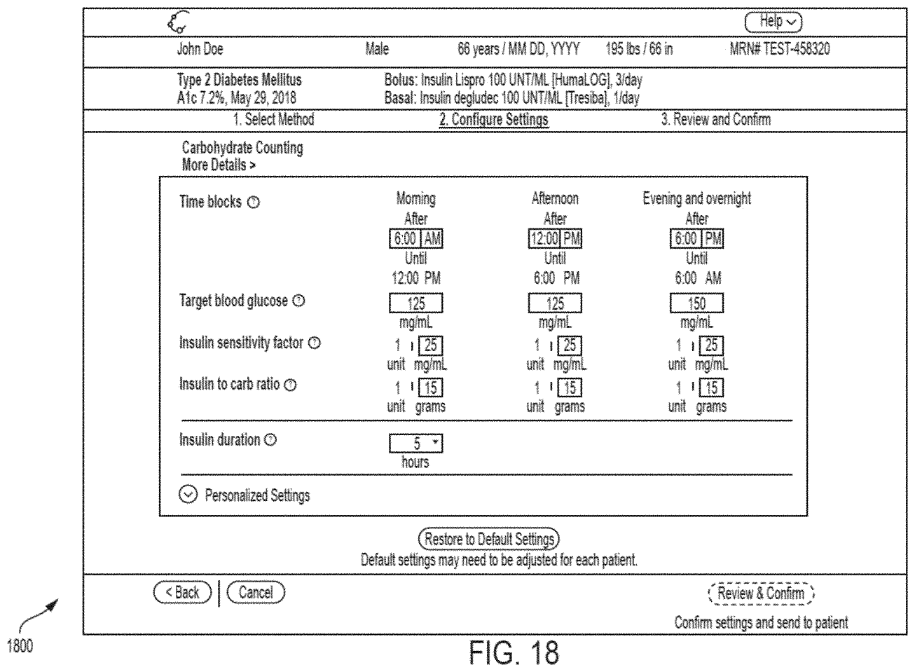

FIG. 18 is a screenshot 1800 depicting a “Configure Settings” tab where the selected dosing regimen is the carbohydrate-bolus-calculator bolus-insulin-dosing algorithm, also referred to simply as the “Carbohydrate Counting” regimen. As shown in FIG. 18, the carbohydrate counting regimens settings tab is similar to the carb meal size regimen settings tab, except that it does not give the HCP user the option to select an expected number of grams of carbohydrates for a “small”, “medium”, and “large” meals.

### II. Example Open-Loop Bolus-Insulin Dosing Algorithms and Associated Regimens

Some or all of the exemplary open-loop bolus-insulin dosing algorithms and associated regimens described herein may calculate a recommended bolus based on Insulin on Board (JOB). IOB is a calculated value that describes the amount of insulin expected to be still active in the patient's body from previously injected bolus insulin doses. This value may reduce the amount of insulin that an algorithm recommends that the patient injects. IOB may be calculated using at least two different approaches.

The first approach may calculate IOB by taking into account only the portion of previous doses that was necessary to correct for a higher-than-desired glucose reading. A single dose of insulin taken at a discrete point in time may be determined based on several parameters, including any or all of (i) an amount of insulin necessary to correct for a higher-than-desired glucose reading, (ii) an additional amount of insulin necessary to “cover” or account for an amount of carbohydrates the patient consumes in a meal shortly after, shortly before, or during the dose, and (iii) any adjustments (positive or negative) necessary to account for lifestyle or health factors that may affect the amount of needed insulin (e.g., whether the patient is ill, whether the patient has, is, or expects to exercise, whether the patient is menstruating, whether the patient is taking steroids, and/or whether the patient is experiencing stress). This first approach may calculate IOB by taking into account only parameter (i), i.e., the amount of insulin necessary to correct for a higher-than-desired glucose reading. The first approach may not factor in either parameters (ii) or (iii) in calculating JOB.

The second approach may calculate IOB by taking into account all insulin that was taken as part of previous doses. In other words, the second approach calculates IOB by taking into account not only parameter (i), but also parameter (ii) (i.e., the amount of insulin necessary to “cover” an amount of carbohydrates in a meal) and also parameter (iii) (any adjustments to account for health factors). This second approach will generally result in a higher calculated JOB, unless negative adjustments from parameter (iii) more than offset the positive contribution from parameter (ii).

Other approaches to calculating IOB are certainly also possible. For example, other approaches may calculate IOB by taking into account parameters (i) and (iii), but not parameter (ii). It is understood that references to Insulin-on-Board or IOB in the following discussion may be calculated using any of the aforementioned approaches. In some embodiments, the HCP, patient, or both may instruct an insulin-dosing algorithm to use a particular approach to calculate IOB. For example, screenshot 1500 in FIG. 15B shows that a HCP may select either a “Conservative” option for calculating Active Insulin (e.g., JOB), or a “Standard” option. The “Standard” option corresponds to the first approach discussed above, while the “Conservative” option corresponds to the second approach. The MMA may include logic capable of calculating IOB using both the first and the second approach, and the patient, HCP, and/or both may be given the option of choosing between the two approaches when calculating a recommended bolus.

A. Daily Titration

An exemplary daily-titration algorithm and associated dosing-regimen is described below.

Recommended doses start from an initial value set by the HCP at the time of dosing regimen prescription. Default starting doses are 10% of total daily basal insulin for breakfast or lunch titrations, and 5% for dinner titrations. Recommended doses for each subsequent day are calculated based on glucose readings logged from the previous day. That is, the recommended doses for the second day are calculated based on glucose readings from the first day; the recommended doses for the third day are calculated based on glucose readings from the second day, and so on. Some exemplary rules for calculating each subsequent day's recommended doses are provided in the table below.

| Breakfast Dose | | Lunch Dose | | Dinner Dose | |
|---|---|---|---|---|---|
| Initial Dose: 10% TBI dose | | Initial Dose: 10% TBI dose | | Initial Dose: 5% TBI dose | |
| Pre-Lunch GL | Dose | Pre-Dinner GL | Dose | Bedtime GL | Dose |
| Reading* | Adjustment | Reading* | Adjustment | Reading* | Adjustment |
| ≥115 | +1 U | ≥115 | +1 U | ≥115 | +1 U |
| 85-114 | no change | 85-114 | no change | 85-114 | no change |
| 56-84 | −1 U | 56-84 | −1 U | 56-84 | −1 U |
| <56 | −2 U | <56 | −2 U | <56 | −2 U |

Abbreviations: GL = glucose level, TBI = total basal insulin, U = units.
*Previous day's GL reading (mg/dL).

The exemplary daily-titration dosing regimen is appropriate for users with T2DM (type-2 diabetes mellitus), between 18 and 85 years old who meet the following criteria:
Failing to achieve glycemic targets despite optimization of basal insulin, with or without metformin
Requiring treatment intensification with mealtime insulin
For whom the set glucose target range of 85-114 mg/dL is appropriate

To enable the exemplary daily-titration dosing regimen, HCPs sets the following parameters:
Total daily basal insulin (TBI), in insulin units
Initial starting dose and meal to titrate, in insulin units

Additionally, the HCP can set the following parameters:
Stable dose(s) per meal(s), in insulin units

The MMA will use the following additional inputs to calculate a recommended dose:
Current GL, in mg/dL: Current GL is used by the MMA to prevent recommending an insulin dose when the Patient has hypoglycemia (glucose ≤70 mg/dL)
Pre-meal GL (GL), in mg/dL: Pre-meal GL is the GL taken following the previous day's titration dose

In at least one embodiment, the exemplary daily-titration dosing regimen does not take correction doses, IOB (Insulin on Board), or adjustment factors as inputs to calculate a recommended dose.

The MMA 568 in at least one embodiment provides recommendations for initiating and titrating basal insulin dosing one meal at a time using glucose values entered by the patient, and is intended to be used in this manner multiple times per day under the direction of an HCP for entering glucose values and receiving dosing recommendations.

B. Fixed Dose

An exemplary fixed-dose algorithm and associated regimen is described below. This fixed-dose algorithm may allow a HCP to set a “fixed” bolus amount per meal, as modified by any adjustment needed to correct for a patient glucose level outside of a desired range.

Meal selection may include no meal (e.g., correction dose only), breakfast, lunch, or dinner. The HCP may assign a fixed dose value to a single mealtime, the same fixed dose value for multiple mealtimes, or different fixed dose values for multiple mealtimes. The dosing regimen may include dose adjustments for insulin on board (JOB), corrections for glucoses levels (GLs) above or below GL target, and lifestyle or health factors (e.g., exercise, stress, illness, steroids, or menstruation).

The exemplary fixed-dose regimen may be appropriate for users with type-1 diabetes mellitus (T1DM) or type-2 diabetes mellitus (T2DM) who are new to bolus therapy or in need of bolus dose optimization. In some cases, the user may require multiple daily injections of mealtime insulin with a rapid-acting insulin analog.

To enable the exemplary fixed-dose regimen, HCPs may set the following parameters:
Fixed Dose (FD), in insulin units per patient-selected meal type
Target GL (GL_{T}), in mg/dL
Insulin Sensitivity Factor (ISF), in mg/dL/insulin unit
Insulin Duration (ID), in hours

In some embodiments, the HCP can control whether or not users are able to alter their regimen-specific settings. For example, a user may not be able to alter their insulin-to-carb ratio unless such a change was enabled by the HCP. And certainly other examples could be listed here as well. Additionally, the HCP in some embodiments can enable any, all, or none of the following lifestyle modifiers and set the corresponding adjustment amount. If enabled, lifestyle modifiers allows a MMA patient user to indicate, via, for example, their mobile device's user interface, whether they are experiencing certain conditions that may affect the amount of insulin their bodies require—such conditions include, for example: whether he/she is exercising or doing a physical activity, whether he/she is experiencing stress, whether he/she is ill, whether he/she is taking steroids, or whether she is menstruating. For each of these lifestyle modifiers, the HCP sets the adjustment amount in percent. The percent value is converted to a decimal value for use in an equation. If the HCP does not enable a modifier, the dosing regimen will use a value of 0.
Activity factor (AF), in percent
Stress factor (SF), in percent
Illness factor (IF), in percent
Steroid factor (StF), in percent
Menstruation factor (MF), in percent

The MMA will use the following additional inputs to calculate a recommended dose:
Meal selection (no meal, breakfast, lunch, dinner) to determine FD value, in insulin units
Current GL (GL), in mg/dL
Current time (t), in hours
Note: current time may include hours, minutes, and seconds, but may be used in the calculation as a fraction of hours

The Insulin-on-Board (IOB), i.e., all insulin doses that are still active at the current time period, will be incorporated in the calculation. A previous dose is considered “active” if it was taken less than ID (Insulin Duration) hours ago. In some embodiments, previous doses may be calculated using the following parameters:
Delivered Bolus (DB), in insulin units
Time of delivered bolus (t_{DB}), in hours
Multiplication factor (X)
   X=0 when an individual delivered bolus was administered less than and including 0.5 hours previous
   X=1 when an individual delivered bolus was administered greater than 0.5 hours and less than ID hours previous

As discussed previously, the DB parameter in the IOB calculation may be defined in one of two ways for a specific previously-administered insulin dose. According to these two methods, the DB parameter may be set as either: (i) only the glucose correction portion of the previous dose, or (ii) the full amount of insulin injected in the previous dose. Option (i) corresponds to the first approach discussed above, and is also referred to herein as the “Standard” option in FIG. 15B. Under this option, the glucose correction portion of the previous dose may be calculated as GL_{DB}−GL_{T}/ISF, where GL_{DB} is the GL entered at the time when the previous dose was calculated, GL_{T} is the target GL, and ISF is the insulin-sensitivity factor (discussed below). Option (ii) corresponds to the second approach discussed above, and is also referred to herein as the “Conservative” option in FIG. 15B. The HCP may select which of these two methods are used to calculate the DB parameter, as shown in FIG. 15B. Alternatively, the patient, or both the HCP and the patient, may have the option to specify which method is to be used to calculate the DB parameter.

In accordance with the exemplary fixed-dose regimen, a bolus insulin dose recommendation (RD) may be calculated using the following formula:

$$R D = ( F D + \frac{G L - G {L}_{T}}{ISF} ) * ( 1 + A F + SF + IF + S t F + M F ) - \sum ( ( 1 - X * \frac{( t - {t}_{D B} ) - 0.5}{ID - 0.5} ) * D B )$$

$$\frac{G L - G {L}_{T}}{ISF}$$

The term FD represents the fixed-dose portion of the formula. The term represents the correction dose portion of the formula, e.g., the portion of the formula that adjusts the recommended dose based on the amount by which the patient's glucose level (GL) exceeds a target glucose level (GL_{T}). ISF is the insulin-sensitivity factor, which represents how much one unit of insulin (e.g., rapid-acting insulin) is expected to drop an individual's glucose level. Generally, to correct a high glucose level, one unit of insulin is needed to drop the glucose level by 50 mg/dl. However, this drop in glucose level can also range from 15-100 mg/dl or more, depending on individual insulin sensitivities, and other circumstances. The term (1+AF+SF+IF+StF+MF) accounts for the previously discussed lifestyle factors. The term

$$Σ ( ( 1 - X * \frac{( t - {t}_{D B} ) - 0.5}{ID - 0.5} ) * D B )$$

accounts for IOB from all previous doses.

The following considerations apply to the formula:
The fixed dose value will be 0 if the patient selects no meal; however, the patient will still receive a correction dose, if applicable.
The correction dose

$$( \frac{G L - G {L}_{T}}{ISF} )$$

may be a positive, negative, or 0 value.
Each IOB calculation

$$( ( 1 - X * \frac{( t - {t}_{D B} ) - 0.5}{ID - 0.5} ) * D B )$$

may be positive or 0. During the calculation, if the IOB calculation returns a negative value, the MMA will use a value of 0 active insulin units.

If the calculated recommended dose returns a negative value, then the MMA will recommend 0 units of insulin. Alternatively, the MMA may recommend to the patient to eat a meal or a certain amount of carbohydrates if the MMA detects that the patient is in or is about to enter a hypoglycemic state, and/or if the MMA determines that the patient has too much IOB (insulin-on-board, i.e., the amount of insulin within the patient's system). In some embodiments, the MMA may recommend that the patient inject glucagon or some other medication that boosts the patient's glucose levels if the MMA determines the patient is in or about to enter a hypoglycemic state.

This exemplary fixed-dosing regimen may reduce the complexity of bolus insulin dosing by allowing the user to receive dose recommendations based on the fixed dosing regimen, as configured by the HCP. If the user has accepted a dosing-regimen prescription, the user will enter the app and can choose to enter the bolus dose recommendation workflow. They will select a meal, enter and/or confirm their glucose measurement, select any additional modifiers such as change in activity or stress, and receive a bolus dose recommendation. The fixed dosing regimen accounts for the HCP-assigned amount of insulin to cover the meal, a correction dose based on current and target blood sugars, and insulin on board based on internal insulin PKPD data.

C. Carbohydrate Bolus Calculator

An exemplary carbohydrate-bolus-calculator algorithm and associated regimen is described below.

The exemplary carbohydrate-bolus-calculator regimen is based on rules for “covering” the carbohydrate content of a meal, and this method is recommended in clinical guidelines as an individualized way to match prandial insulin to meal intake. Accordingly, it can be used at any time of day, and may not be tied to a specific meal. It calculates a recommended bolus insulin dose based on insulin sensitivity, carbohydrate intake, GL targets, and current GL. It will also account for correction doses, JOB, and optional adjustment factors.

A bolus can be calculated based on two factors: (i) insulin required for food or carbohydrate coverage and (ii) insulin required to correct for high blood sugar.

The bolus dose for food or carbohydrate coverage is prescribed based on an insulin-to-carbohydrate ratio (ICR). The insulin-to-carbohydrate ratio represents how many grams of carbohydrate are covered or disposed of by 1 unit of insulin. Generally, one unit of rapid-acting insulin will dispose of 12-15 grams of carbohydrate; however, this parameter can vary from 4-30 grams or more of carbohydrate depending on an individual's sensitivity to insulin. Insulin sensitivity can vary according to the time of day, from person to person, and is affected by physical activity and stress.

The exemplary carbohydrate-bolus-calculator regimen is appropriate for users with T1DM or T2DM, who accurately carb count or who can learn to do so in the judgment of the HCP. In some cases, the users may require multiple daily injections of mealtime insulin with a rapid-acting insulin analog.

To enable the exemplary carbohydrate-bolus-calculator regimen, HCPs may set the following parameters:
Target GL (GL_{T}), in mg/dL
Insulin Sensitivity Factor (ISF), in mg/dL/insulin unit
Insulin to Carb Ratio (ICR), in grams carbohydrate/insulin units
Insulin Duration (ID), in hours

Additionally, the HCP can enable any, all, or none of the following lifestyle modifiers and set the corresponding adjustment amount. The HCP sets the lifestyle factors adjustments using percentages that is appropriate for their patient. The percent value is converted to a decimal value for use in an equation. If the HCP does not enable a modifier, the dosing regimen will use a value of 0.
Activity factor (AF), in percent
Stress factor (SF), in percent
Illness factor (IF), in percent
Steroid factor (StF), in percent
Menstruation factor (MF), in percent

The MMA will use the following additional inputs to calculate a recommended dose:
Current GL (GL), in mg/dL
Current time (t), in hours
Carb intake (CHO), in grams carbohydrate, OR meal insulin (MI), in insulin units

The Insulin-on-Board (IOB), i.e., all insulin doses that are still active at the current time period, will be incorporated in the calculation. A previous dose is considered “active” if it was taken less than ID (Insulin Duration) hours ago. In some embodiments, previous doses may be calculated using the following parameters:
Delivered Bolus (DB), in insulin units
Time of delivered bolus (t_{DB}), in hours
Multiplication factor (X)
   X=0 when an individual delivered bolus was administered less than and including 0.5 hours previous
   X=1 when an individual delivered bolus was administered greater than 0.5 hours and less than ID hours previous

As discussed previously, the DB parameter in the IOB calculation may be defined in multiple ways. For example, the DB parameter may be set as either (i) full amount of insulin from the previous dose, or (ii) only the glucose correction portion of the previous dose.

The MMA user may choose to input either carb intake (CHO) (e.g., an expected number of carbohydrates in a meal) or meal-time insulin (MI) (e.g., expressed in units of insulin). If the MMA user chooses to input meal-time insulin, the MMA user may input a number of units of insulin that the user expects will cover a particular meal. For instance, if the MMA user does not know exactly how many grams of carbohydrates are in a particular meal, but knows from experience that a certain number of units of insulin should be sufficient to cover that type of meal, the MMA user may elect to input a number of units of meal-time insulin instead of expected carb intake. Depending on whether the MMA user enters carb intake or meal insulin, the appropriate formula below will be used to calculate the dose recommendation.

A bolus insulin dose recommendation (RD) will be calculated using the following formula when the user enters a carb intake value:

$$R D = ( \frac{CHO}{ICR} + \frac{G L - G {L}_{T}}{ISF} ) * ( 1 + A F + SF + IF + S t F + M F ) - \sum ( ( 1 - X * \frac{( t - {t}_{D B} ) - 0.5}{ID - 0.5} ) * DB )$$

$$where ( \frac{CHO}{ICR} )$$

represents the carbohydrate intake portion of the formula.

A bolus insulin dose recommendation will be calculated using the following formula when the user enters a meal insulin value:

$$R D = ( MI + \frac{G L - G {L}_{T}}{ISF} ) * ( 1 + A F + SF + IF + S t F + M F ) - \sum ( ( 1 - X * \frac{( t - {t}_{D B} ) - 0.5}{ID - 0.5} ) * D B )$$

where MI represents the insulin to cover meal intake.

The following considerations apply to the formula:
The correction dose

$$( \frac{G L - G {L}_{T}}{ISF} )$$

may be a positive, negative, or 0 value.
Each IOB calculation

$$( ( 1 - X * \frac{( t - {t}_{D B} ) - 0.5}{ID - 0.5} ) * D B )$$

may be positive or 0. During the calculation, if the IOB calculation returns a negative value, the MMA will use a value of 0 active insulin units.

If the calculated recommended dose returns a negative value, then the MMA will recommend 0 units of insulin. Alternatively, the MMA may recommend to the patient to eat a meal or a certain amount of carbohydrates if the MMA detects that the patient is in or is about to enter a hypoglycemic state, and/or if the MMA determines that the patient has too much IOB (insulin-on-board, i.e., the amount of insulin within the patient's system). In some embodiments, the MMA may recommend that the patient inject glucagon or some other medication that boosts the patient's glucose levels if the MMA determines the patient is in or about to enter a hypoglycemic state.

The exemplary carbohydrate-bolus-calculator regimen helps to reduce the complexity of bolus insulin dosing by allowing the user to receive dose recommendations based on the bolus calculator regimen, as configured by the HCP. If the user has accepted a dosing-regimen prescription, the user will enter the app and can choose to enter the bolus dose recommendation workflow. They will enter and/or confirm their glucose-level measurement, enter the number of carbohydrates that they intend to consume, select any additional modifiers such as change in activity or stress, and receive a bolus dose recommendation. The user may elect to enter the number of insulin units needed to cover a meal instead of the amount of carbohydrates, using the regimen to help with correction doses and accounting for insulin on board. The carbohydrate bolus calculator dosing regimen accounts for the amount of insulin to cover the intended carb intake, a correction dose based on current and target blood sugars, and insulin on board based on internal insulin PKPD data.

D. Carbohydrate Meal-Size-Based Calculator

An exemplary carbohydrate-meal-size-based-calculator algorithm and associated regimen is described below. The exemplary carbohydrate-meal-size-based-calculator regimen utilizes the functionality of the exemplary carbohydrate-bolus-calculator regimen described above, with additional settings made by the HCP for small, medium and large qualitative assessments of the carbohydrate meal content. For example, instead of having the MMA user input an expected number of carbs in a meal, or an amount of mealtime insulin, the MMA user may simply indicate whether he/she is about to have a “small”, “medium”, or “large” meal. The MMA user's selection is then translated into an estimated number of carbohydrates based on pre-set values (potentially configurable by the HCP and/or the MMA user) for a “small”, “medium”, or “large” meal.

The exemplary carbohydrate-meal-size-based-calculator regimen is appropriate for users with T1DM and T2DM, who can estimate the size of a meal, and in particular the size of the carbohydrate content in a meal. In some cases, the user may require multiple daily injections of mealtime insulin with a rapid-acting insulin analog.

To enable the exemplary carbohydrate-meal-size-based-calculator regimen, HCPs may set the following parameters:
Target GL (GL_{T}), in mg/dL
Insulin Sensitivity Factor (ISF), in mg/dL/insulin unit
Insulin to Carb Ratio (ICR), in grams carbohydrate/insulin units
Insulin Duration (ID), in hours
Estimated amount of carbs in a “small” meal size, in grams carbohydrate
Estimated amount of carbs in a “medium” meal size, in grams carbohydrate
Estimated amount of carbs in a “large” meal size, in grams carbohydrate

Additionally, the HCP can enable any, all, or none of the following lifestyle modifiers and set the corresponding adjustment amount. The HCP sets the lifestyle factors adjustments using percentages that is appropriate for their patient. The percent value is converted to a decimal value for use in the equation. If the HCP does not enable a modifier, the dosing regimen will use a value of 0.
Activity factor (AF), in percent
Stress factor (SF), in percent
Illness factor (IF), in percent
Steroid factor (StF), in percent
Menstruation factor (MF), in percent

The MMA will use the following additional inputs to calculate a recommended dose:
Current GL (GL), in mg/dL
Current time (t), in hours
Carb intake meal size (MS), as small, medium, large, or none selection
   The MMA will replace meal size with the corresponding number of grams of carbohydrate based on the HCP pre-set for the selected meal
   The MMA will replace meal size with a value of 0 if Patient selects the no meal option

The Insulin-on-Board (IOB), i.e., all insulin doses that are still active at the current time period, will be incorporated in the calculation. A previous dose is considered “active” if it was taken less than ID (Insulin Duration) hours ago. In some embodiments, previous doses may be calculated using the following parameters:
Delivered Bolus (DB), in insulin units
Time of delivered bolus (t_{DB}), in hours
Multiplication factor (X)
   X=0 when an individual delivered bolus was administered less than and including 0.5 hours previous
   X=1 when an individual delivered bolus was administered greater than 0.5 hours and less than ID hours previous

As discussed previously, the DB parameter in the IOB calculation may be defined in multiple ways. For example, the DB parameter may be set as either (i) full amount of insulin from the previous dose, or (ii) only the glucose correction portion of the previous dose.

A bolus insulin dose recommendation (RD) will be calculated using the following formula:

$$R D = ( \frac{M S}{ICR} + \frac{G L - G {L}_{T}}{ISF} ) * ( 1 + A F + SF + IF + S t F + M F ) - \sum ( ( 1 - X * \frac{( t - {t}_{D B} ) - 0.5}{ID - 0.5} ) * DB )$$

$$where ( \frac{MS}{ICR} )$$

represents the carbohydrate intake.

The following considerations apply to the formula:
The correction dose

$$( \frac{G L - G {L}_{T}}{ISF} )$$

may be a positive, negative, or 0 value.
Each IOB calculation

$$( ( 1 - X * \frac{( t - {t}_{D B} ) - 0.5}{ID - 0.5} ) * D B )$$

may be positive or 0. During the calculation, if the IOB calculation returns a negative value, the MMA will use a value of 0 active insulin units.

If the calculated recommended dose returns a negative value, then the MMA will recommend 0 units of insulin. Alternatively, the MMA may recommend to the patient to eat a meal or a certain amount of carbohydrates if the MMA detects that the patient is in or is about to enter a hypoglycemic state, and/or if the MMA determines that the patient has too much IOB (insulin-on-board, i.e., the amount of insulin within the patient's system). In some embodiments, the MMA may recommend that the patient inject glucagon or some other medication that boosts the patient's glucose levels if the MMA determines the patient is in or about to enter a hypoglycemic state.

The exemplary carbohydrate meal-size-based calculator regimen may help to reduce the complexity of bolus insulin dosing by allowing the user to receive dose recommendations based on the meal-size-based-calculator regimen, as configured by the HCP. If the user has accepted a dosing-regimen prescription, the user will enter the app and can choose to enter the bolus-dose-recommendation workflow. They will enter and/or confirm their glucose measurement, select their meal size, select any additional modifiers such as change in activity or stress, and receive a bolus-dose recommendation. The carbohydrate-meal-size-based calculator dosing regimen accounts for the amount of insulin to cover intended amount of carb intake, a correction dose based on current and target blood sugars, and insulin on board based on internal insulin PKPD data.

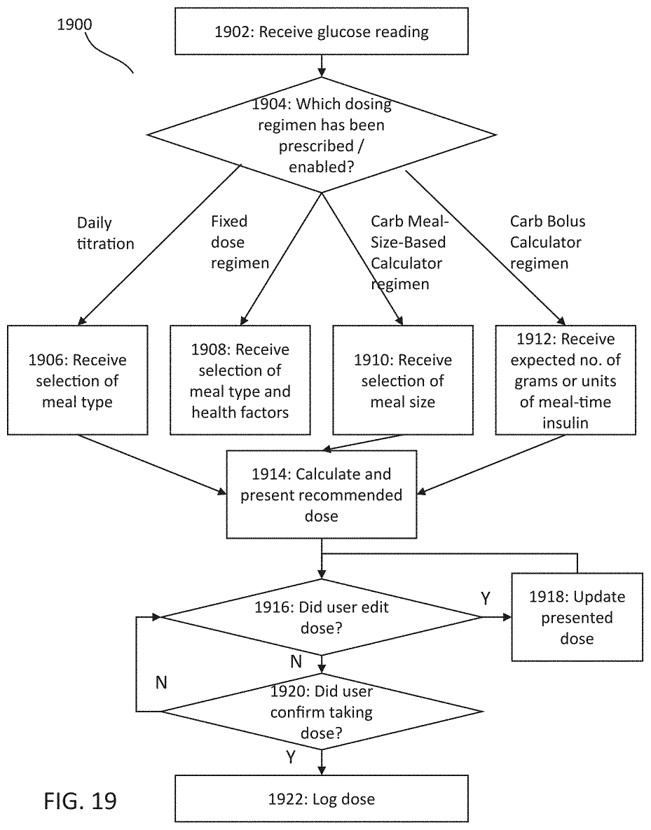

FIG. 19 is a flowchart illustrating an exemplary process 1900 implemented by a MMA running on a mobile device (e.g., MMA 568 running on mobile device 104, or MMA 570 running on mobile device 532). The MMA uses process 1900 to gather information regarding the patient's condition and to recommend an insulin dose, according to whichever insulin dosing regimen has been prescribed and enabled on the MMA.

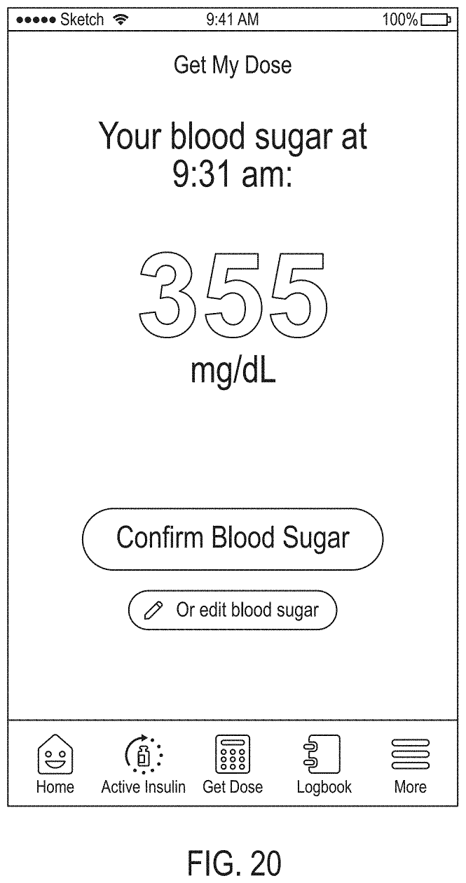

At step 1902, the MMA receives a glucose reading from the patient. This may be done in a variety of ways. For example, FIG. 20 depicts an exemplary screenshot from the MMA in which the MMA automatically synchronizes with a patient's connected glucose sensor, such as a continuous glucose meter (CGM). As shown in FIG. 20, the MMA may depict the time at which the glucose meter reading was taken from the connected glucose meter (in this example, 9:31 am), the blood glucose level (in this case, 355 mg/dL), and also allow the patient to edit the blood sugar reading by touching the button labeled “Or edit blood sugar.” If the patient agrees with the glucose level received from the connected glucose meter, or if the patient has finished editing the glucose level, the patient may touch the button labeled “Confirm Blood Sugar”, at which point the MMA will record the presented glucose level in the patient's log.

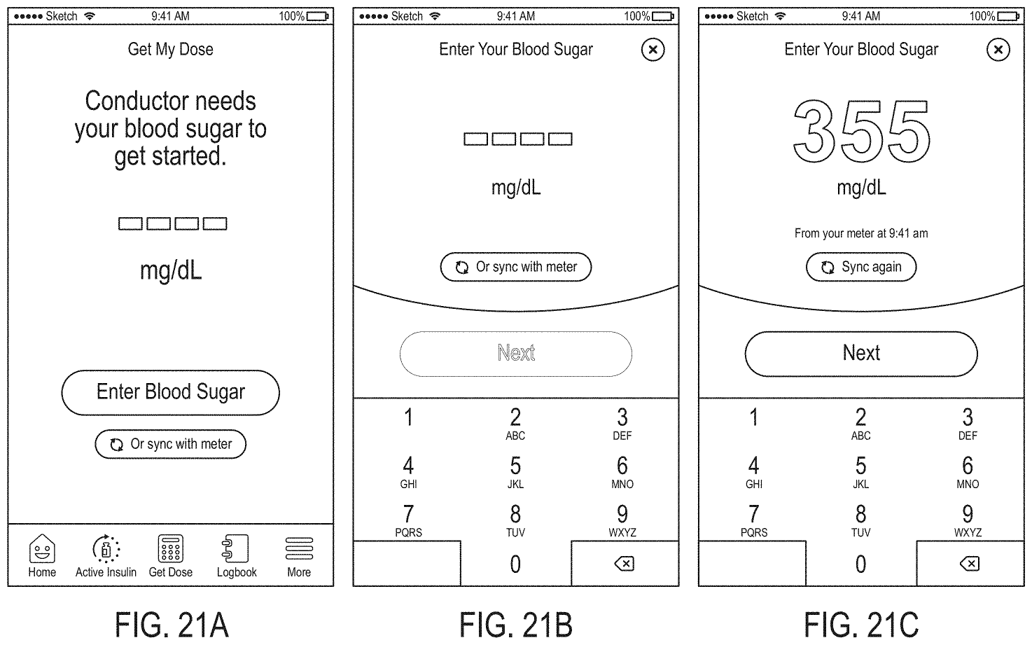

Alternatively, the MMA may allow the patient to manually type in his or her glucose level, or prompt the MMA to synchronize with a connected glucose meter. These methods of receiving the patient's glucose level are illustrated in FIGS. 21A, 21B, and 21C. In FIG. 21A, the MMA prompts the patient to input his or her glucose level. If the patient touches the button labeled “Enter Blood Sugar”, the MMA transitions to the screen shown in FIG. 21B, where the patient can manually type in his or her glucose level. Once the patient types in the glucose level, the patient may touch the “Next” button to instruct the MMA to record the input glucose level in the patient's log. If the patient touches the button labeled “Or sync with meter”, the MMA transitions to the screen shown in FIG. 21C, where the MMA synchronizes with the patient's connected glucose meter (e.g., a BGM, CGM, or FGM). In this case, as shown in FIG. 21C, the MMA receives a glucose level of 355 mg/dL from the patient's connected glucose meter. Alternatively, if the patient touches the button “Or sync with meter” in FIG. 21B, the MMA will also transition to the screen shown in FIG. 21C. Once the MMA receives the glucose level from the connected meter, the patient can instruct the MMA to record the glucose level in the patient's log by touching the “Next” button.

Returning to FIG. 19, once the MMA receives a glucose reading, the MMA moves to step 1904. At this point, the MMA determines which dosing regimen has been prescribed and has been enabled on the MMA. If the daily titration regimen has been prescribed/enabled, process 1900 branches to step 1906. If the fixed dose regimen has been prescribed/enabled, process 1900 branches to step 1908. If the carb bolus calculator regimen has been prescribed/enabled, process 1900 branches to step 1910. If the carb meal-size-based calculator regimen has been prescribed/enabled, process 1900 branches to step 1912.

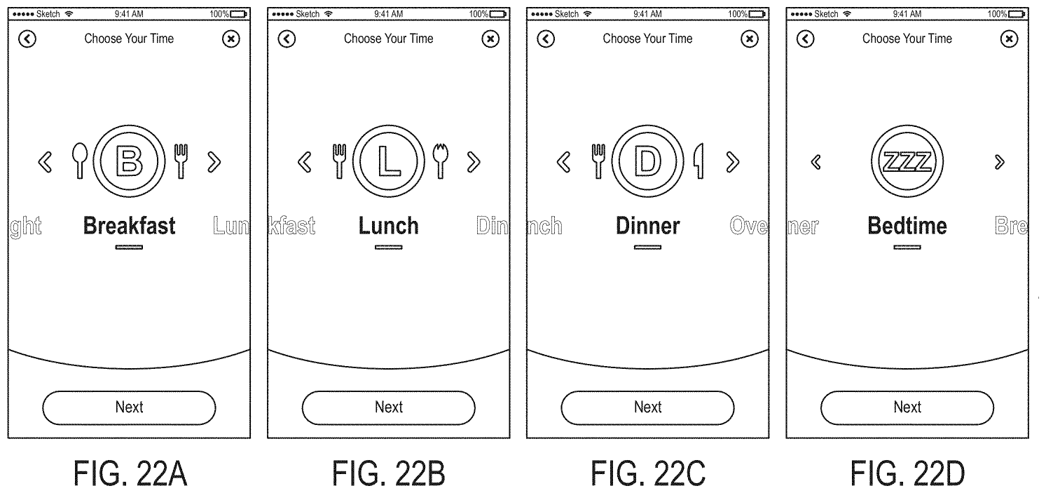

At step 1906 (used for the daily titration regimen), the MMA receives a selection of meal type or time of day from the patient. For example, the illustrative screenshots shown in FIGS. 22A-22C allow the patient to indicate whether he or she is about to take a dose at breakfast time (FIG. 22A), at lunch time (FIG. 22B), at dinner time (FIG. 22C), or at bedtime (FIG. 22D). The MMA uses this information to calculate a recommended insulin dose, according to the exemplary algorithms described above for the “Daily Titration” dosing-regimen.

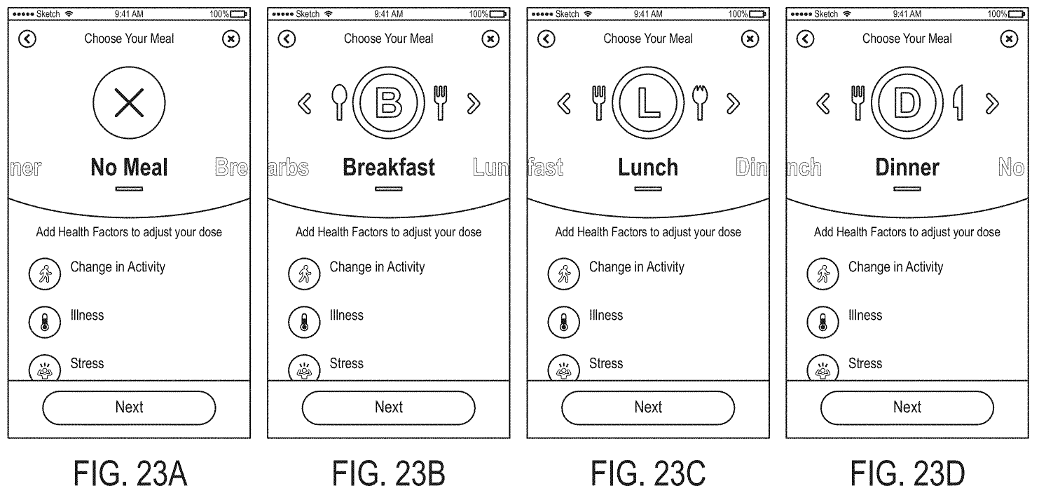

At step 1908 (used for the fixed-dose regimen), the MMA receives a selection of meal type as well as any health factors that apply from the patient. For example, the illustrative screenshots shown in FIG. 23A-23D allow the patient to indicate whether he or she is about to take a dose that is not associated with any meal (FIG. 23A), at breakfast time (FIG. 23B), at lunch time (FIG. 23C), or at dinner time (FIG. 23D). Regardless of which meal selection the patient chooses, the patient may also choose any health factors that the patient is currently experiencing, such as stress, activity, illness, steroids, and/or menstruation. The MMA uses this information to calculate a recommended insulin dose, according to the exemplary algorithms described above for the “Fixed-Dose” dosing-regimen.

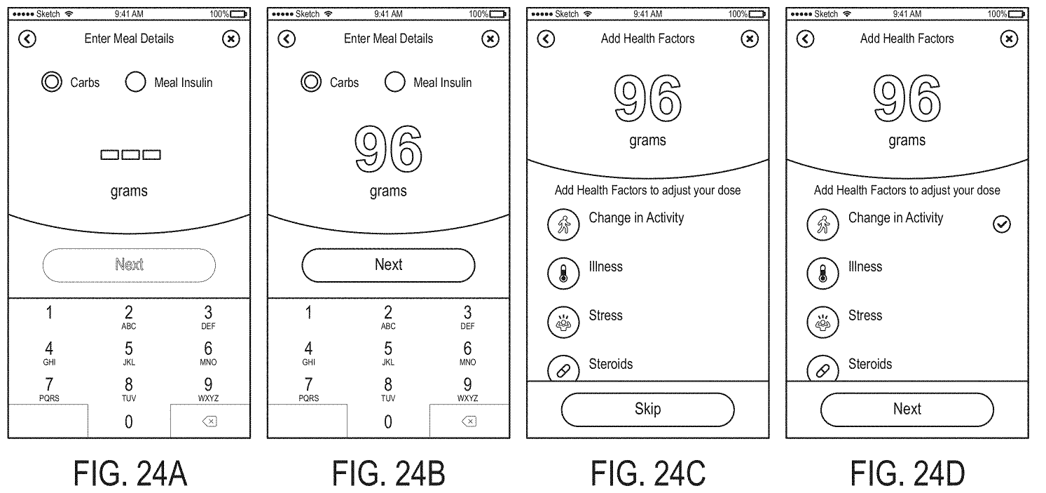

At step 1910 (used for the carb bolus calculator regimen), the MMA receives an expected number of grams of carbohydrates that the patient intends to consume, and/or an expected number of units of meal-time insulin that the patient predicts will be sufficient to cover the carbohydrates he or she intends to consume. The MMA also receives any health factors that apply from the patient. For example, FIG. 24A prompts the patient to input an expected number of grams of carbohydrates to be consumed. The patient can input an estimated number of carbohydrates using the numbered keypad. As depicted in FIG. 24B, the patient has indicated that he or she intends to consume 96 grams of carbohydrates. When the patient presses the “Next” button, the MMA transitions to the screen shown in FIG. 24C, where the patient can choose any health factors that he or she is currently experiencing, such as stress, activity, illness, steroids, and/or menstruation. Once the patient has finished choosing health factors, the patient may touch a “Next” button (not shown) which appears when at least one health factor has been chosen. Alternatively, the patient may press the “Skip” button if the patient is not experiencing any of these health factors. If at least one health factor is chosen, or if the patient presses the “skip” button, the MMA transitions to the screen shown in FIG. 24D. In FIG. 24D, the MMA displays the selected number of grams of carbohydrates as well as any selected health factors for the patient's confirmation. The patient may confirm by touching the “Next” button.

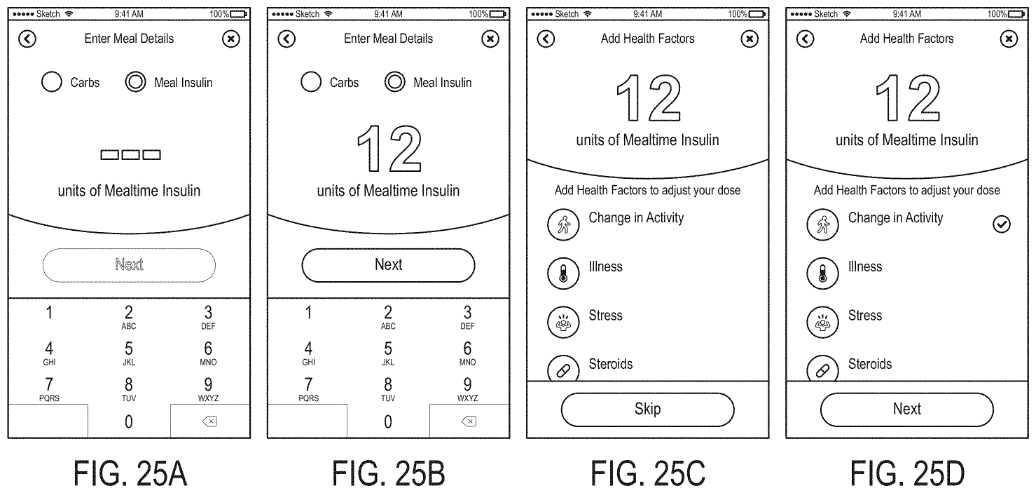

Alternatively, the patient can decide to input a number of units of meal-time insulin that the patient expects (based, for instance, on experience) will cover the meal that he or she is about to consume. The patient may indicate to the MMA that he or she will input meal-time insulin by selecting the “Meal Insulin” radio button on FIG. 24A. If the “Meal Insulin” radio button is selected, the MMA transitions to the series of screens shown in FIG. 25A-D. In FIG. 25A, the MMA prompts the patient to input a number of units of meal-tine insulin using the numbered keypad. For instance, as depicted in FIG. 25B, the patient has input 12 units of meal-time insulin. When the patient presses the “Next” button, the MMA transitions to the screen shown in FIG. 25C, where the patient can choose any health factors that he or she is currently experiencing, such as stress, activity, illness, steroids, and/or menstruation. Alternatively, the patient may press the “Skip” button if the patient is not experiencing any of these health factors. In FIG. 25D, the MMA displays the selected number of units of meal-time insulin as well as any selected health factors for the patient's confirmation. The patient may confirm by pressing the “Next” button.

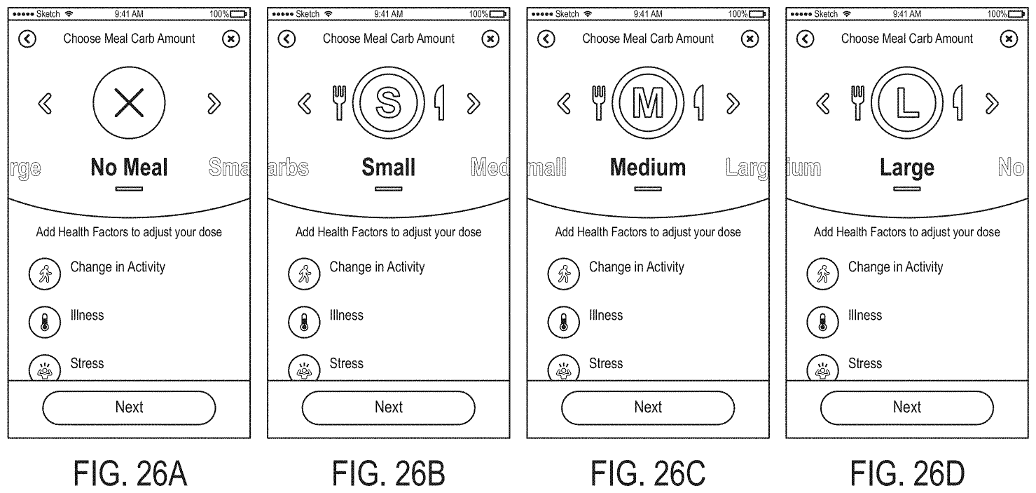

Returning to FIG. 19, at step 1912 (used for the carb meal-size-based calculator regimen), the MMA receives a selection of meal size, as well as any health factors that apply from the patient. For example, the illustrative screenshots shown in FIGS. 26A-D allow the patient to indicate whether he or she is about to take a dose that is not associated with any meal (FIG. 26A), with a “small” meal (FIG. 26B), with a “medium”-sized meal (FIG. 26C), or with a “large”-sized meal (FIG. 26D). These meal-size selections are translated to an expected number of carbohydrates according to the pre-set meal-size definitions described previously. Regardless of which meal-size selection the patient chooses, the patient may also choose any health factors that the patient is currently experiencing, such as stress, activity, illness, steroids, and/or menstruation. The MMA uses this information to calculate a recommended insulin dose, according to the exemplary algorithms described above for the “Carbohydrate Meal-Size-Based Calculator” regimen.

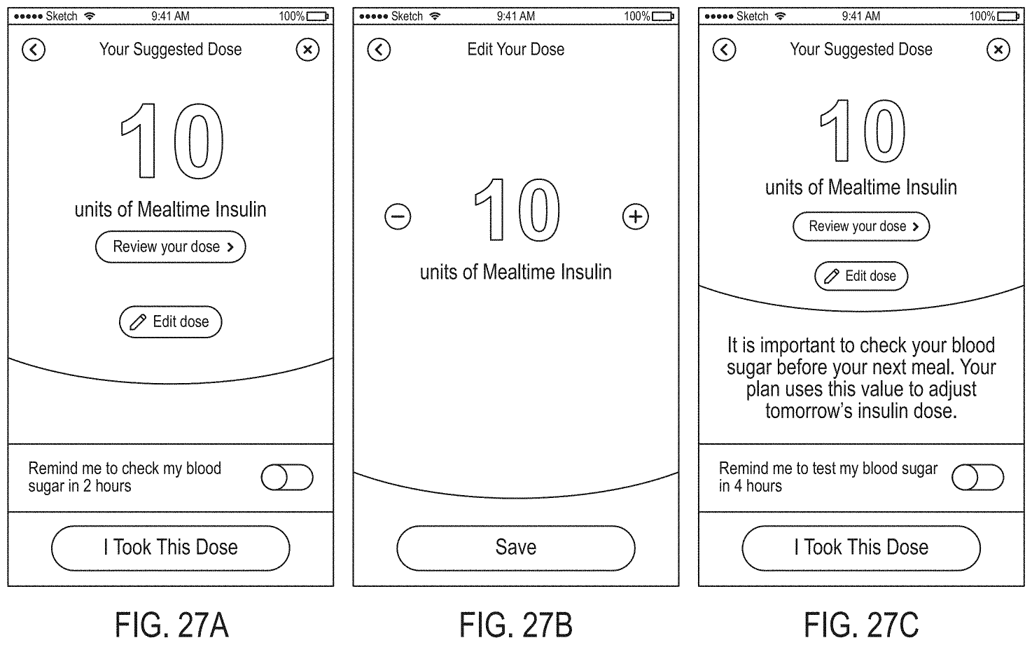

Returning to FIG. 19, after gathering information from the patient in steps 1906, 1908, 1910, or 1912, the MMA branches to step 1914 where it calculates and presents a recommended dose of insulin. The algorithms for calculating a recommended dose of insulin vary according to which dosing regimen is currently prescribed and enabled, and has been described previously. The MMA then presents this dose to the user, as illustrated in the exemplary screenshot in FIG. 27A (which, in this example, recommends a dose of 10 units of meal-time insulin). The patient will also be given an opportunity to edit the recommended dose by, for instance, touching the “Edit dose” button in FIG. 27A.

At step 1916, the MMA determines whether the user edited the recommended dose. If so, the MMA branches to step 1918, where it updates the presented dose. This process is illustrated in FIG. 27B, where the patient may increase the recommended dose by hitting the “+” button to the right of the presented dose, or decrease the recommended dose by hitting the “−” button to the left of the presented dose. When the patient has finished editing the recommended dose, the patient may touch the “Save” button, which takes the patient back to the screen shown in FIG. 27A. If the daily titration dosing regimen has been prescribed/enabled, the MMA may optionally display the additional message shown in FIG. 27C, which reminds the patient that it is important to check his or her blood sugar before the patient's next meal, as the daily titration dosing regimen uses this value to adjust tomorrow's insulin dose.

If the patient has not edited the presented dose, the MMA branches to step 1920, where it determines whether the user has confirmed taking the dose. If not, the MMA branches back to step 1916. If so, the MMA proceeds to step 1922, where it logs the dose. This process is illustrated in FIGS. 27A and 27C, where the patient can confirm that they took the dose by touching the button labeled “I Took This Dose.” Touching this button causes the MMA to log the presented dose in the patient's logbook.

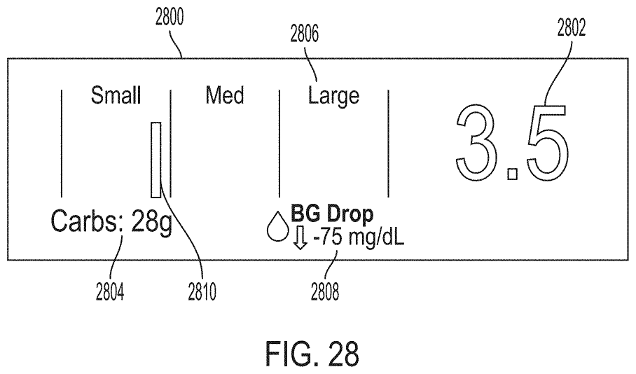

FIG. 28 depicts an alternative user-interface display 2800 that presents a dose of insulin to be administered to a patient, currently being administered to the patient, and/or recently administered to the patient. The dose of insulin may have been recommended by one of the insulin dosing regimens discussed previously, or may have been manually input or adjusted by the patient. User-interface display 2800 may be displayed on the MMA in lieu of or in addition to the screenshots depicted in FIGS. 27A-27C. Alternatively, or in addition, user-interface display 2800 may be displayed on a drug-delivery device, such as on a visual screen mounted on an insulin pen.

User-interface display 2800 comprises a plurality of panels. A panel may comprise data displayed by a processor on a portion of a visual display screen. A first panel 2802 displays a number of units of insulin to be administered to the patient, currently being administered to the patient, or recently administered to the patient—in the example depicted, the first panel 2802 shows 3.5 units of insulin.

A second panel 2804 displays an amount carbohydrates expected to be covered by the number of units of insulin displayed by the first panel. As used in the specification and claims herein, the amount X of carbohydrates “expected to be covered by” a certain number of units of insulin Y shall correspond to an estimated amount of carbohydrates that, if ingested by a patient with diabetes, would require administration of Y units of insulin to the patient in order to keep the patient's glucose level after ingestion of said amount X of carbohydrates the same as the patient's glucose level before ingestion of said amount X of carbohydrates. In the example depicted, the second panel 2804 indicates that 28 g of carbohydrates are expected to be covered by the presented 3.5 units of insulin. The number of carbohydrates displayed by the second panel 2804 may be calculated by dividing the number of units of insulin displayed by the first panel 2802 by an insulin-to-carbohydrate ratio (ICR). The user-interface display 2800 may use a default ICR value that applies to all patients, or to a patient-specific ICR provided by the patient, a caregiver, or a HCP. In some embodiments, the ICR may both be specific to a patient as well as to a time-of-day, if the ICR for a particular patient is known to vary according to a predictable pattern according to time of day. In this example, if the ICR being used by the user-interface display 2800 is 0.125, the number of grams of carbohydrates displayed by the second panel 2804 (28 g) may be derived by dividing the number of units of insulin displayed by the first panel 2802 (3.5 units) by the ICR of 0.125.

A third panel 2806 indicates whether the number of carbohydrates displayed by the second panel corresponds to a small meal size or a large meal size. In the example depicted in FIG. 28, this third panel 2806 takes the form of a spectrum that displays a range of meal sizes between a “small” meal size, a “medium” meal size, or a “large” meal size, and also includes an indicator 2810 that indicates where the number of carbohydrates displayed by the second panel 2804 falls on this spectrum. In other embodiments (not shown), the third panel 2806 may simply display a letter, word, or symbol that indicates a meal-size classification. In the simplest case, meals may be simply classified into one of two categories: “small” or “large.” In some embodiments, meals may be classified into one of three categories: “small”, “medium”, or “large.” In yet other embodiments, meals may be classified into four or more meal-size classifications.

The third panel 2806 may be calculated based on tunable parameters that define different meal-size classifications. For instance, in embodiments that classify meals into three categories (e.g., “small”, “medium”, or “large”), such tunable parameters may include a minimum carbohydrate threshold for a “medium”-sized meal (e.g., 30 g) and a maximum carbohydrate threshold for a “medium”-sized meal (e.g., 60 g). Any carbohydrate value that falls between this minimum and maximum carbohydrate threshold may be classified as a “medium”-sized meal. Any carbohydrate value that falls below the minimum carbohydrate threshold may be classified as a “small”-sized meal, and any carbohydrate value that falls above the maximum carbohydrate threshold may be classified as a “large”-sized meal. Panels that include a spectrum that display a range (as depicted in FIG. 28) may also determine where to place the indicator based on these meal size definitions, e.g., by scaling the placement of the indicator along the spectrum depending on the meal size definitions. As discussed previously, these meal size definitions may be customizable by the patient, a caregiver, or a HCP.

A fourth panel 2808 displays an expected drop in the patient's glucose level that results from administration of the number of units of insulin displayed by the first panel 2802. In the example depicted, the fourth panel 2808 indicates that a blood glucose drop of 75 mg/dL is expected to result from administration of the 3.5 units of insulin displayed in the first panel 2802. The glucose drop displayed by the fourth panel 2808 may be calculated by multiplying the number of units of insulin displayed by the first panel 2802 by an insulin sensitivity factor (ISF). The user-interface display 2800 may use a default ISF that applies to all patients, or to a patient-specific ISF provided by the patient, a caregiver, or a HCP. In some embodiments, the ISF may both be specific to a patient as well as to a time-of-day, if the ISF for a particular patient is known to vary according to a predictable pattern according to time of day. In this example, if the ISF is 21.4, the expected BG drop displayed by the fourth panel 2808 (75 mg/dL) may be derived by multiplying the number of units displayed by the first panel 2802 (3.5 units) by the ISF of 21.4.

Although FIG. 28 displays a user-interface display 2800 that includes all four panels 2802, 2804, 2806 and 2808, some embodiments may display only a subset of these four panels, or may display additional panels. For instance, some embodiments may include only the first panel 2802, the second panel 2804, and the third panel 2806, and may not display the fourth panel 2808. In other embodiments, the user-interface display 2800 may include only the first panel 2802 and the fourth panel 2808, and may not display the second panel 2804 or the third panel 2806. In yet other embodiments, the user-interface display 2800 may include only the first panel 2802 and the second panel 2804. The panels need not be arranged in the order, configuration, or relative sizes displayed in FIG. 28.

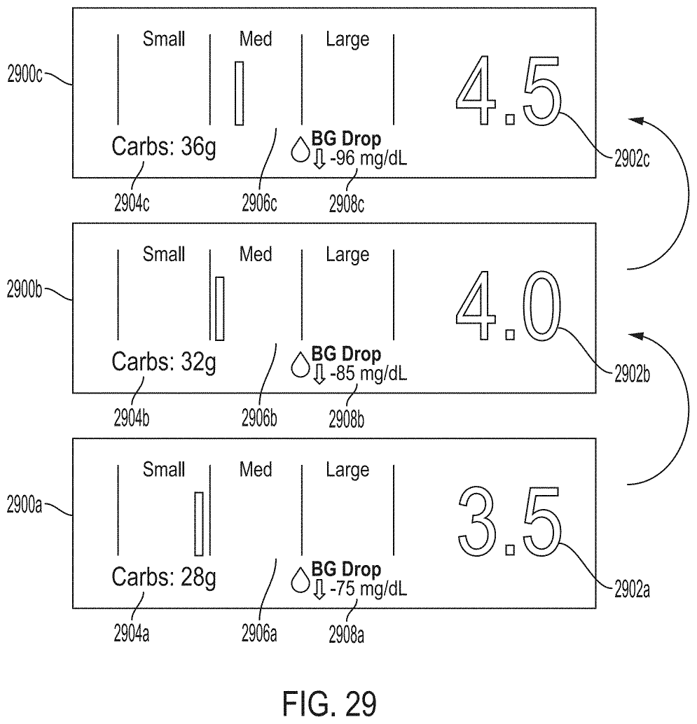

Regardless of which specific panels are displayed, all displayed panels may be updated as the user adjusts the number of units of insulin to be administered, currently being administered, or that was recently administered. FIG. 29 shows a sequence of user-interface displays as the number of units of insulin is adjusted from 3.5 units in display 2900a to 4.0 units in display 2900b and to 4.5 units in display 2900c. As shown, the number of units displayed in the first panel changes from 3.5 in panel 2902a to 4.0 in panel 2902b and then to 4.5 in 2902c. Similarly, the number of carbohydrates displayed in the second panel (see panels 2904a, 2904b, and 2904c), the meal size indicator in the third panel (see panels 2906a, 2906b, and 2906c), and the expected glucose drop in the fourth panel (see panels 2908a, 2908b, and 2908c) automatically update as the number of units of insulin changes.

Although the example shown in FIG. 29 shows an example where the number of units of insulin is adjusted in increments of 0.5 units, this need not be the case. Generally, the granularity by which the number of units may be adjusted on the disclosed user-interface may depend upon the dose resolution of the delivery device with which the user-interface is associated. If, for example, a delivery device (such as an insulin pen) is capable of delivering different doses in 1.0 unit increments, the user-interface may also be configured to allow for dose adjustments in 1.0 unit increments. If the delivery device is capable of delivering different doses in 2.0 unit increments, the user-interface may also be configured to allow for adjustments in 2.0 unit increments. The same is the case for other delivery-device dose resolutions, such as 0.1 units, 5.0 units, or any other dose resolution. Aligning the user-interface's dose resolution to conform to the dose resolution of the patient's delivery device allows the patient to easily decide how to set a dose on his or her delivery device. In some cases, the user-interface may be designed to work with multiple types of delivery devices having different dose resolutions. In such embodiments, the dose resolution on the user-interface may be configurable by a patient, a caregiver or a HCP to conform to the dose resolution on a particular patient's delivery device.

Displaying the number of covered carbohydrates, meal size indication, and/or expected glucose drop at the same time as a number of units of insulin to be administered/currently being administered/recently administered provides multiple benefits to patients. Patients often administer insulin in order to cover a certain number of carbohydrates, to cover a certain meal size, or to achieve a specific drop in glucose level. By displaying some or all of this information to the patient, the patient can easily tell whether the displayed dose will achieve his or her goals and can adjust the displayed dose accordingly.

Displaying this information simultaneously also solves a technical problem present in prior-known delivery device user-interfaces by reducing over- or under-dosing due to rounding error. For instance, when patients attempt to calculate a dose to cover a specific meal, patients often start by guessing at the number of carbohydrates in the meal before them. Once patients arrive at an estimated number of carbohydrates, patients then calculate a dose size based on the estimated number of carbs. In calculating the dose size, however, patients often round to the nearest unit or half-unit of insulin, depending on their drug-delivery device's dose resolution. This process requires the patient to go through two rounding or estimating steps to arrive at a dose of insulin: a first step in which the patient estimates the number of carbohydrates in a meal, and a second step in which the patient converts that carbohydrate estimate into a number of units of insulin. This two-step process increases the risk of over- or under-estimating the dose needed to cover a specific meal.

This is best illustrated using an exemplary scenario. Consider a case where a patient estimates that he is about to consume a meal with 75 g of carbohydrates. The patient arrives at this estimate based solely on the visual appearance of the meal and his prior experience. The user then calculates a dose based on an insulin-to-carbohydrate ratio of 8, which results in a dose of 75 g/8=9.375 units. However, due to his drug delivery device's dose resolution, he can only administer whole units of insulin, and so rounds down and administers only 9 units. In this case, if the patient had under-estimated the number of carbs in the meal (e.g., if the meal before the patient actually contained 80 g of carbs), and then had rounded down, the patient may have doubly underestimated the dose needed to cover the meal. If not compensated for, this under-dosing of insulin can make it more difficult for the patient to achieve his glycemic goals.

By displaying the number of carbohydrates to be covered as well as the number of units of insulin, the user-interface display decreases errors introduced due to rounding. By dialing between 9 units and 10 units on the user-interface, the patient can quickly determine that 9 units would cover 72 grams of carbohydrates, while 10 units would cover 80 grams of carbohydrates. The user can then estimate whether the meal before him is closer to 72 grams or 80 grams of carbohydrates. In contrast to the previously-discussed scenario which required two calculation or estimation steps, this calculation involves only a single, simple estimation step by the patient. This increases the chance that the patient will arrive at the correct dose to cover the meal before him. For example, faced with a choice between 72 grams and 80 grams, the patient may be more likely to decide that the meal before him is actually closer to 80 grams and may choose to administer 10 units of insulin.

The disclosed user-interface can also decrease rounding errors in other scenarios as well, including situations where the patient is making dosing decisions based not on carbohydrates but on meal size, and/or target drop in glucose level. By displaying the meal size and/or estimated drop in glucose level along with the presented insulin dose, the disclosed user-interface similarly reduces the number of estimation/rounding steps required by the patient, and thus increases the chances that the patient will arrive at the correct dose to cover a particular meal or to achieve a particular glucose level.

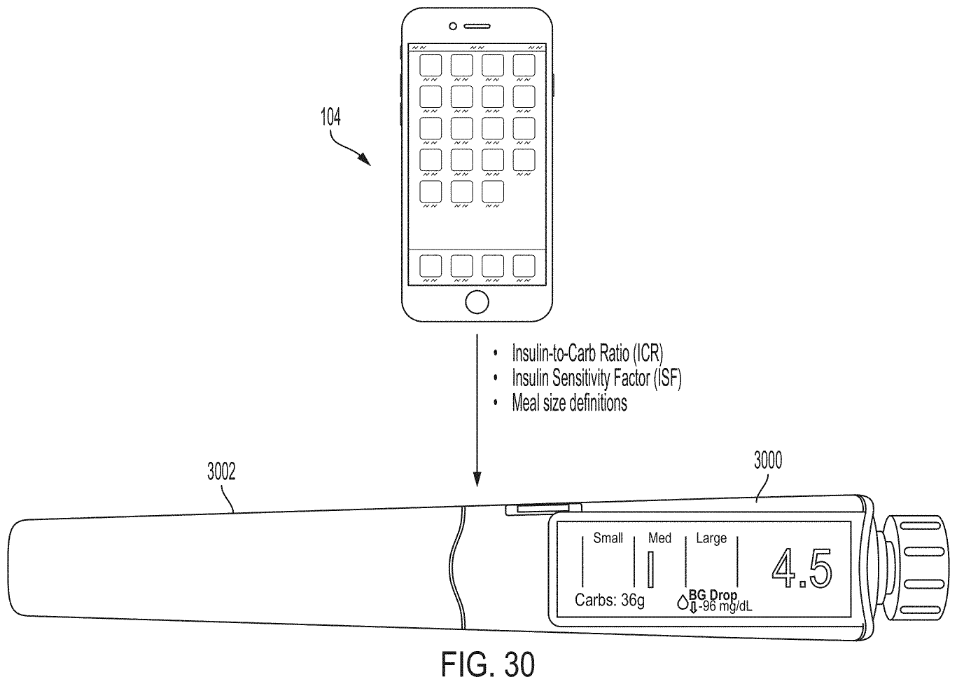

As previously discussed, the disclosed user-interface 2800 may be displayed on the MMA executing on a mobile device in lieu of or in addition to the previously disclosed screenshots in FIGS. 27A-27C. In addition, however, the disclosed user-interface 2800 may also be displayed on a mobile drug-delivery device, such as a connected insulin pen. One such exemplary implementation is depicted in FIG. 30, which shows a connected insulin pen 3002 having a screen 3000 that displays the previously-discussed user-interface. Such a connected insulin pen 3002 may be in wireless communication with a mobile device, such as mobile device 104, via a Bluetooth, WiFi, NFC, or other wireless link. Through this wireless link, the connected insulin pen 3002 may receive various parameters for computing information displayed by the panels in the user-interface, such as an insulin-to-carb ratio (ICR), an insulin sensitivity factor (ISF), and/or meal-size definitions, such as a minimum and a maximum carbohydrate threshold for a “medium”-sized meal (as discussed previously).

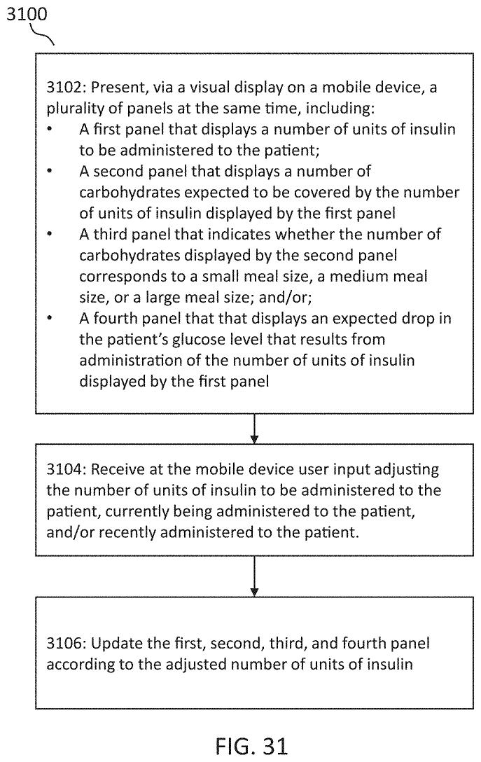

FIG. 31 depicts a flow-chart illustrating an exemplary process executed by a mobile device (such as a smartphone or a drug-delivery device). At step 3102, the mobile device presents, via a visual display on the mobile device, a plurality of panels at the same time. These panels may include a first panel that displays a number of units of insulin to be administered to the patient, a second panel that displays a number of carbohydrates expected to be covered by the number of units of insulin displayed by the first panel, a third panel that indicates whether the number of carbohydrates displayed by the second panel corresponds to a small meal size, a medium meal size, or a large meal size, and/or a fourth panel that displays an expected drop in the patient's glucose level that results from administration of the number of units of insulin displayed by the first panel. As previously discussed, some embodiments may not display all four panels but only a subset of these panels. Some embodiments may also display additional panels.

At step 3104, the mobile device receives user input adjusting the number of units of insulin to be administered to the patient, currently being administered to the patient, and/or recently administered to the patient. For example, this user input may be received via “plus” or “minus” buttons on the screen of a MMA similar to those discussed above in FIG. 27B, or using a numeric keypad. Alternatively, the user input may be received by the user dialing a knob on a connected insulin-delivery device, such as an insulin pen.

At step 3106, the mobile device updates the first panel, the second panel, the third panel, and/or the fourth panel according to the adjusted number of units of insulin. As previously described, this adjustment may be done using parameters that convert a number of units of insulin to a number of covered carbohydrates (such as an insulin-to-carbohydrate ratio, or ICR), that convert a number of units of insulin to an expected drop in glucose level (such as an insulin sensitivity factor, or ISF), or that convert a number of covered carbohydrates to a meal size.

While this invention has been described as having an exemplary design, the embodiments of the present disclosure may be further modified within the spirit and scope of this disclosure. This application is therefore intended to cover any variations, uses, or adaptations of the disclosed embodiments using its general principles.

1. A method comprising:
presenting, via a healthcare-professional (HCP)-portal application, multiple medication-dosing regimens pertaining to a patient, wherein each presented medication-dosing regimen is associated with a respective different medication-dosing algorithm;
receiving, via the HCP-portal application, an HCP selection of at least one of the presented medication-dosing regimens; and
transmitting, to a medical mobile application (MMA) on a mobile device associated with the patient, HCP-selected-regimen data indicative of the at least one HCP-selected medication-dosing regimen.

2. The method of claim 1, wherein:
the presented medication-dosing regimens comprise one or more insulin-dosing regimens; and
the at least one HCP-selected medication-dosing regimen comprises at least one HCP-selected insulin-dosing regimen.

3. The method of claim 2, wherein:
the one or more presented insulin-dosing regimens comprise at least one or more basal-insulin-dosing regimens and one or more bolus-insulin-dosing regimens.

4. (canceled)

5. (canceled)

6. The method of claim 2, wherein the at least one HCP-selected insulin-dosing regimen comprises a daily-titration bolus-insulin-dosing regimen associated with a daily-titration bolus-insulin-dosing algorithm.

7. The method of claim 2, wherein the at least one HCP-selected insulin-dosing regimen comprises a fixed-dose bolus-insulin-dosing regimen associated with a fixed-dose bolus-insulin-dosing algorithm.

8. The method of claim 2, wherein the at least one HCP-selected insulin-dosing regimen comprises a carbohydrate-bolus-calculator bolus-insulin-dosing regimen associated with a carbohydrate-bolus-calculator bolus-insulin-dosing algorithm.

9. The method of claim 2, wherein the at least one HCP-selected insulin-dosing regimen comprises a carbohydrate-meal-size-based-calculator bolus-insulin-dosing regimen associated with a carbohydrate-meal-size-based-calculator bolus-insulin-dosing algorithm.

10. The method of claim 1, further comprising receiving, from the MMA, a regimen-accepted indication indicating that the MMA had received, via a mobile-device user interface of the mobile device, a patient-acceptance indication associated with the at least one HCP-selected medication-dosing regimen.

11. The method of claim 1, further comprising receiving, from the MMA, a regimen-rejected indication indicating that the MMA had received, via a mobile-device user interface of the mobile device, a patient-rejection indication associated with the at least one HCP-selected medication-dosing regimen, and disabling the MMA on the mobile device associated with the patient in response to the patient-rejection indication.

12. The method of claim 1, wherein the HCP-selected-regimen data is operable to unlock the at least one HCP-selected medication-dosing regimen on the MMA.

13. The method of claim 12, wherein the MMA is operable to access implementation data prestored on the mobile device for the at least one HCP-selected medication-dosing regimen only after the at least one HCP-selected medication-dosing regimen has been unlocked by the HCP-selected-regimen data.

14. The method of claim 12, wherein the MMA is operable to download implementation data for the at least one HCP-selected medication-dosing regimen onto the mobile device only after the at least one HCP-selected medication-dosing regimen has been unlocked by the HCP-selected-regimen data.

15. The method of claim 12, wherein the HCP-selected-regimen data is operable to unlock the at least one HCP-selected medication-dosing regimen on the MMA conditionally upon receipt by the MMA of a patient-acceptance indication with respect to the at least one HCP-selected medication-dosing regimen.

16. (canceled)

17. The method of claim 1, wherein the MMA is configured to provide, via a mobile-device user interlace of the mobile device, dosing recommendations based on the at least one HCP-selected medication-dosing regimen.

18. A connected-care server system comprising:
one or more servers, each server comprising one or more communication interfaces, one or more processors, and data storage,
wherein the data storage of the one or more servers collectively contain instructions that, when executed by one or more of the processors of the one or more servers, are operable to cause the connected-care server system to carry out a set of server-system functions, the set of server-system functions including:
presenting, via a healthcare-professional (HCP)-portal application in communication with at least one of the communication interfaces of the one or more servers, multiple medication-dosing regimens pertaining to a patient, wherein each presented medication-dosing regimen is associated with a respective different medication-dosing algorithm;
receiving, via the HCP-portal application, an HCP selection of at least one of the presented medication-dosing regimens; and
transmitting, via at least one of the communication interfaces of the one or more servers, to a medical mobile application (MMA) on a mobile device associated with the patient, HCP-selected-regimen data indicative of the at least one HCP-selected medication-dosing regimen.

19. The system of claim 18, wherein:
the presented medication-dosing regimens comprise one or more insulin-dosing regimens; and
the at least one HCP-selected medication-dosing regimen comprises at least one HCP-selected insulin-dosing regimen.

20. The system of claim 19, wherein:
the one or more presented insulin-dosing regimens comprises at least one or more basal-insulin-dosing regimens and one or more bolus-insulin-dosing regimens.

21. (canceled)

22. (canceled)

23. The system of claim 19, wherein the at least one HCP-selected insulin-dosing regimen comprises a daily-titration bolus-insulin-dosing regimen associated with a daily-titration bolus-insulin-dosing algorithm.

24. The system of claim 19, wherein the at least one HCP-selected insulin-dosing regimen comprises a fixed-dose bolus-insulin-dosing regimen associated with a fixed-dose bolus-insulin-dosing algorithm.

25. The system of claim 19, wherein the at least one HCP-selected insulin-dosing regimen comprises a carbohydrate-bolus-calculator bolus-insulin-dosing regimen associated with a carbohydrate-bolus-calculator bolus-insulin-dosing algorithm.

26. The system of claim 19, wherein the at least one HCP-selected insulin-dosing regimen comprises a carbohydrate-meal-size-based-calculator bolus-insulin-dosing regimen associated with a carbohydrate-meal-size-based-calculator bolus-insulin-dosing algorithm.

27. The system of claim 18, the set of server-system functions further comprising receiving, from the MMA, a regimen-accepted indication indicating that the MMA had received, via a mobile-device user interface of the mobile device, a patient-acceptance indication associated with the at least one HCP-selected medication-dosing regimen.

28. The system of claim 18, the set of server-system functions further comprising receiving, from the MMA, a regimen-rejected indication indicating that the MMA had received, via a mobile-device user interface of the mobile device, a patient-rejection indication associated with the at least one HCP-selected medication-dosing regimen, and disabling the MMA on the mobile device associated with the patient in response to the regimen-rejected indication.

29. The system of claim 18, wherein the HCP-selected-regimen data is operable to unlock the at least one HCP-selected medication-dosing regimen on the MMA.

30. The system of claim 29, wherein the MMA is operable to access implementation data prestored on the mobile device for the at least one HCP-selected medication-dosing regimen only after the at least one HCP-selected medication-dosing regimen has been unlocked by the HCP-selected-regimen data.

31. The system of claim 29, wherein the MMA is operable to download implementation data tor the al least one HCP-selected medication-dosing regimen onto the mobile device only after the at least one HCP-selected medication-dosing regimen has been unlocked by the HCP-selected-regimen data.

32. The system of claim 29, wherein the HCP-selected-regimen data is operable to unlock the at least one HCP-selected medication-dosing regimen on the MMA conditionally upon receipt by the MMA of a patient-acceptance indication with respect to the at least one HCP-selected medication-dosing regimen.

33. (canceled)

34. The system of claim 18, wherein the MMA is configured to provide, via a mobile-device user interface of the mobile device, dosing recommendations based on the at least one HCP-selected medication-dosing regimen.

35. One or more non-transitory computer-readable media (CRM) having stored thereon instructions that, when executed by one or more processors, are operable to cause the one or more processors to carry out a set of functions, the set of functions comprising:
presenting, via a HCP-portal application, multiple medication-dosing regimens pertaining to a patient, wherein each presented medication-dosing regimen is associated with a respective different medication-dosing algorithm;
receiving, via the HCP-portal application, an HCP selection of at least one of the presented medication-dosing regimens; and
transmitting, to a medical mobile application (MMA) on a mobile device associated with the patient, HCP-selected-regimen data indicative of the at least one HCP-selected medication-dosing regimen.

36. The CRM of claim 35, wherein:
the presented medication-dosing regimens comprise one or more insulin-dosing regimens; and
the at least one HCP-selected medication-dosing regimen comprises at least one HCP-selected insulin-dosing regimen.

37. The CRM of claim 36, wherein:
the one or more presented insulin-dosing regimens comprises at least one or more basal-insulin-dosing regimens and one or more bolus-insulin-dosing regimens.

38. (canceled)

39. (canceled)

40. The CRM of claim 36, wherein the at least one HCP-selected insulin-dosing regimen comprises a daily-titration bolus-insulin-dosing regimen associated with a daily-titration bolus-insulin-dosing algorithm.

41. The CRM of claim 36, wherein the at least one HCP-selected insulin-dosing regimen comprises a fixed-dose bolus-insulin-dosing regimen associated with a fixed-dose bolus-insulin-dosing algorithm.

42. The CRM of claim 36, wherein the at least one HCP-selected insulin-dosing regimen comprises a carbohydrate-bolus-calculator bolus-insulin-dosing regimen associated with a carbohydrate-bolus-calculator bolus-insulin-dosing algorithm.

43. The CRM of claim 36, wherein the at least one HCP-selected insulin-dosing regimen comprises a carbohydrate-meal-size-based-calculator bolus-insulin-dosing regimen associated with a carbohydrate-meal-size-based-calculator bolus-insulin-dosing algorithm.

44. The CRM of claim 35, the set of functions further comprising receiving, from the MMA, a regimen-accepted indication indicating that the MMA had received, via a mobile-device user interface of the mobile device, a patient-acceptance indication associated with the at least one HCP-selected medication-dosing regimen.

45. The CRM of claim 35, the set of functions further comprising receiving, from the MMA, a regimen-rejected indication indicating that the MMA had received, via a mobile-device user interface of the mobile device, a patient-rejection indication associated with the at least one HCP-selected medication-dosing regimen, and disabling the MMA on the mobile device associated with the patient in response to the patient-rejection indication.

46. The CRM of claim 35, wherein the HCP-selected-regimen data is operable to unlock the at least one HCP-selected medication-dosing regimen on the MMA.

47. The CRM of claim 46, wherein the MMA is operable to access implementation data prestored on the mobile device for the at least one HCP-selected medication-dosing regimen only after the at least one HCP-selected medication-dosing regimen has been unlocked by the HCP-selected-regimen data.

48. The CRM of claim 46, wherein the MMA is operable to download implementation data for the at least one HCP-selected medication-dosing regimen onto the mobile device only after the at least one HCP-selected medication-dosing regimen has been unlocked by the HCP-selected-regimen data.

49. The CRM of claim 46, wherein the HCP-selected-regimen data is operable to unlock the at least one HCP-selected medication-dosing regimen on the MMA conditionally upon receipt by the MMA of a patient-acceptance indication with respect to the at least one HCP-selected medication-dosing regimen.

50. (canceled)

51. The CRM of claim 35, wherein the MMA is configured to provide, via a mobile-device user interface of the mobile device, dosing recommendations based on the at least one HCP-selected medication-dosing regimen.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. The method, of claim 1, further comprising:
presenting, by the MMA a plurality of panels simultaneously on a visual display of the mobile device, wherein the plurality of panels comprise:
a first panel that displays a number of units of insulin to be administered to patient, and
a second panel that displays an amount of carbohydrates expected to be covered by the number of units of insulin displayed by the first panel;
receiving at the mobile device user input representative of a requested adjustment to the number of units of insulin displayed by the first panel; and
updating, by the MMA, each panel of the plurality of panels according to the requested adjustment to the number of units of insulin displayed by the first panel, wherein the updating includes displaying an adjusted number of units of insulin by the first panel and an adjusted amount of carbohydrates expected to be covered by the adjusted number of units of insulin with the second panel.

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)
